SEQUENCE LISTING (32 sequences)

SEQ ID NO: 1 - DNA, 1305 bp, Setaria italica
atggcgcctcggcggcgccgggcgtggcctgcggctgctgccgtcgttacggccgtggtcaccgcggcggcggccgtcgtcttcgcggcccaggccgcggacatggtccaggagaggctgacggtgggcatgacgatcgtcgccgacgccgcgttcacgggggcagtgtgcctcgacgggagcccaccggcgtaccacctgcaccgcggctccggcgccggcgcgcgcagctggctgctccagttcgagggcggcggctggtgcaacgacgtgcggtcgtgcgccgagagggccgggacgcgccgagggtccacccacctcatgaccaaggtcgaggtcttctccggcatcctcagcaacctcccggccatgaatccagatttttacaactggaaccgtgtgaagctacgttattgcgatggtgggtccttctcgggtgattcagcatacataaatgactcctcagtcctctacttcagaggtcagagaatatgggatgctattatcactgacctactacagaagggactagcgaaagctgaaaattgtaaatctgaaattcacttcttttatgctttcaaatcttgggtttgcgtacttcaggtactgctctcaggttgttcagcaggaggcctagctacattcttccactgcgacagcctcaaggagcgtcttggaggagctaccacggtgaaatgcctgagtgatgcgggatttttccttgatctgaatgatatttctggcatcaacaatataaggcaattctttagcagcctagtctccgtgcagggagttcagaagaatttgaacaaagattgccagaattcaacagactatccatacctgtgtttttttccgcaatatgcacttccatacattagaaccccatatttcatcttgaattcagcttatgacgtgtatcagttccatcacattttcgtgccaccttcatctgatcctaggggacagtggagccgctgtaagatggatcctggtgcatgcagcacatcccaaattgcaaccctccaaggactgagaaatgcaatgttgacatctctgaaacagattgaaggcgaaccagaggcagggatgttcataaactcttgctttgcacattgccagagtgagctgcaagacacatggtttgcaccagattccccaatgatacatgataggaaaattgcagaggtgataggcgattggtactttgaaagagatgctgccaaggagattgactgtgcctatccttgtgattcaacttgtcacaaccttataccatctgatcaggaattgaggatgtattag

SEQ ID NO: 2 - protein, 434 aa, Setaria italica
MAPRRRRAWPAAAAVVTAVVTAAAAVVFAAQAADMVQERLTVGMTIVADAAFTGAVCLDGSPPAYHLHRGSGAGARSWLLQFEGGGWCNDVRSCAERAGTRRGSTHLMTKVEVFSGILSNLPAMNPDFYNWNRVKLRYCDGGSFSGDSAYINDSSVLYFRGQRIWDAIITDLLQKGLAKAENCKSEIHFFYAFKSWVCVLQVLLSGCSAGGLATFFHCDSLKERLGGATTVKCLSDAGFFLDLNDISGINNIRQFFSSLVSVQGVQKNLNKDCQNSTDYPYLCFFPQYALPYIRTPYFILNSAYDVYQFHHIFVPPSSDPRGQWSRCKMDPGACSTSQIATLQGLRNAMLTSLKQIEGEPEAGMFINSCFAHCQSELQDTWFAPDSPMIHDRKIAEVIGDWYFERDAAKEIDCAYPCDSTCHNLIPSDQELRMY

SEQ ID NO: 3 - protein, 394 aa, Populus trichocarpa
MAGSRLGQWPCLVVCILILLKAEGASIPITIIETAVSSGAVCLDGSQPGYHFDKGSGSGIDNWLVHMEGGGWCESVESCVSRRDTFRGSSSKMEKTMGFSGILGSKQAANPDFYNWNRIKVRYCDGSSFTGDVEAVDPETKLYFRGERVWQAVIDELLAKGMQNARNAILSGCSAGGLAAILHCDKFQSLLPATARVKCVSDAGYFIHGTDISGGSRIESFFGQVVKTHGSAKNLPASCTSKMNPELCFFPQYVAQTMRTPLFIINSAYDSWQIKNILAPTAVDSKKEWKDCKLDLKKCSASQLQTVQDYRTQFLKAVDTGLGSSSTRGLWIDSCYAHCQSGSVSTWLNDKSPEVGDTKMGKAVGDWFYDRSAMEKIDCPYSCNPTCVSVDSES

SEQ ID NO: 4 - protein, 399 aa, Vigna radiata
METSRKGQWLSVLICVVLLLKAEGVPVGITFVENAVAKGAVCLDGSPPAYHFHKGSGAGINNWIVHFEGGGWCNNVTSCLSRRDTRLGSSKKMDTQLSFSGFFSNSKKFNPDFYDWNRIKVRYCDGSSFTGDVEAVDPATNLHFRGSRVFAAVVDDLLAKGMKNAQNAIISGCSAGGLAAILNCDRFKSLLPRTTKVKCLADAGYFINVKDVSGAQRIEEFYSQVVQTHGSAKNLPASCTSRLRPGLCFFPQNVVSQISTPIFFVNAAYDSWQIKNILAPGAADPHGTWRECKLDIKKCSSNQLSAMQVFRTDFLRAFGAVGNSPSKGHFIDGCYAHCQTGTQETWLRNDSPVLGSTTIAKAVGDWYYDRKPFKQIDCAYPCNPTCHNRIFDQNERPDV

SEQ ID NO: 5 - protein, 5 aa, artificial
  conserved hydrolase superfamily motif
GXSXG

SEQ ID NO: 6 - DNA, 1864 bp, Setaria italica
atggcgcctcggcggcgccgggcgtggcctgcggctgctgccgtcgttacggccgtggtcaccgcggcggcggccgtcgtcttcgcggcccaggccgcggacatggtccaggagaggctgacggtgggcatgacgatcgtcgccgacgccgcgttcacgggggcaggcaagtccgcgtcgcgcgtccgttttattactccccttctttcccgtggcgatgtccgatgtttgtgtgccagcccgcgacgtcatccgctgcgtttccttcctttctactgctcacagtgtgcctcgacgggagcccaccggcgtaccacctgcaccgcggctccggcgccggcgcgcgcagctggctgctccagttcgagggcggcggctggtgcaacgacgtgcggtcgtgcgccgagagggccgggacgcgccgagggtccacccacctcatgaccaaggtcgaggtcttctccggcatcctcagcaacctcccggccatgaatccaggtacgagctcagtcaccaatcttctcacctcccctctgaaatctgaattttactttgaaactagagaaagtaaacttcagattgctgagtccctatgcccccatgcattcatgtttggacgtgatcgagcgtaaactgtcttgccaagttgccattcactcgagatctcttgcttagctaattcgtcggcagcaatcgacgcatgctccccttcatgattgaacagcttgcttttgcaaaaggtgacagcgatgcggcaatcgcagattcacaaacgggtaggtagcacgaacagttctgctttaatctaggtccgtgcatttccctctcgcgaattcaagctcatgatcctacaaaaatcatttcgtggggtggctggcataagatgcacagtgcctctgcttctggcccacgtacatctgtttcaggcccacgtacatctgtttcaggcccaccgcaaaattcttccggttcaggagagctgaatgcaaatttggactcatttataaaatgtaaaactccaatcatgatcatgaatcttctagtagccatttctacatataaagtttcttctaggggcttcaggtgtactctacagttaatagctagcatgtctgccactgttgatttcttttgtcacagctcaagtcttcatccttttgtagatttattttattttctgttgccacatagtgacaggcctactttacagataaactttatgctttccagtttagcttgaaattcttttatttgcagaaatgtgaattatcaaagcttttcatcatatttggcaaaattaggcatgcatgttaacaccatgttagcaaaaatgttgaacagatttttacaactggaaccgtgtgaagctacgttattgcgatggtgggtccttctcgggtgattcagcatacataaatgacgtatggttctcaagctgacataaaccaagccctaagttaccatgtgtaatttttttttgaaacgaaaaaaataattacacatgagttttctaaagtctgcggcattttgctcactgtttggtgtttgccatttttcagtcctcagtcctctacttcagaggtcagagaatatgggatgctattatcactgacctactacagaagggactagcgaaagctgaaaatgtattttctccaaaaatattgattgtttcactaagtgtaaatctgaaattcacttcttttatgctttcaaatcttgggtttgcgtacttcaggtactgctctcaggttgttcagcaggaggcctagctacattcttccactgcgacagcctcaaggagcgtcttggaggagctaccacggtgaaatgcctgagtgatgcgggatttttccttgatctg

SEQ ID NO: 7 - DNA, 20 bp, artificial
  synthetic oligonucleotide primer
tttagccctgccttcatacg

SEQ ID NO: 8 - DNA, 22 bp, artificial
  synthetic oligonucleotide primer
caagacagtttacgctcgatca

SEQ ID NO: 9 - DNA, 20 bp, artificial
  synthetic oligonucleotide primer
aactggaaccgtgtgaagct

SEQ ID NO: 10 - DNA, 21 bp, artificial
  synthetic oligonucleotide primer
caacgtgcacaacagaattga

SEQ ID NO: 11 - DNA, 21 bp, artificial
  synthetic oligonucleotide primer
cttggtgagctgcagagatgg

SEQ ID NO: 12 - DNA, 21 bp, artificial
  synthetic oligonucleotide primer
agggtctcgaacttcttgatg

SEQ ID NO: 13 - DNA, 20 bp, artificial
  synthetic oligonucleotide primer
tttagccctgccttcatacg

SEQ ID NO: 14 - DNA, 21 bp, artificial
  synthetic oligonucleotide primer
ggattccagagaaggcaatct

SEQ ID NO: 15 - DNA, 20 bp, artificial
  synthetic oligonucleotide primer
acctccagagggttgttcat

SEQ ID NO: 16 - DNA, 21 bp, artificial
  synthetic oligonucleotide primer
caacgtgcacaacagaattga

SEQ ID NO: 17 - DNA, 21 bp, artificial
  synthetic oligonucleotide primer
cgatgtaggagggcgtggata

SEQ ID NO: 18 - DNA, 19 bp, artificial
  synthetic oligonucleotide primer
cttctgcgggcgatttgtg

SEQ ID NO: 19 - DNA, 20 bp, artificial
  synthetic oligonucleotide primer
gatgtccgatgtttgtgtgc

SEQ ID NO: 20 - DNA, 20 bp, artificial
  synthetic oligonucleotide primer
ctcgaccttggtcatgaggt

SEQ ID NO: 21 - DNA, 26 bp, artificial
  synthetic oligonucleotide primer
cttcataggaatggaagctgcgggta

SEQ ID NO: 22 - DNA, 26 bp, artificial
  synthetic oligonucleotide primer
cgaccaccttgatcttcatgctgcta

SEQ ID NO: 23 - DNA, 20 bp, artificial
  synthetic oligonucleotide primer
gcggtggccgaacaagggaa

SEQ ID NO: 24 - DNA, 21 bp, artificial
  synthetic oligonucleotide primer
ccagccgctcgcatctttcca

SEQ ID NO: 25 - DNA, 21 bp, artificial
  synthetic oligonucleotide primer
atttcatgaccttctaccacc

SEQ ID NO: 26 - DNA, 21 bp, artificial
  synthetic oligonucleotide primer
cactccagcaacatctttctc

SEQ ID NO: 27 - DNA, 21 bp, artificial
  synthetic oligonucleotide primer
tcatgcacttcgatcttaccc

SEQ ID NO: 28 - DNA, 21 bp, artificial
  synthetic oligonucleotide primer
tggcaccagaatgttccttac

SEQ ID NO: 29 - DNA, 20 bp, artificial
  synthetic oligonucleotide primer
caagatttggagatcccgtg

SEQ ID NO: 30 - DNA, 19 bp, artificial
  synthetic oligonucleotide primer
aatgctccacggcgaacag

SEQ ID NO: 31 - DNA, 23 bp, artificial
  synthetic oligonucleotide primer
tcgatgctcaccctgttgtttgg

SEQ ID NO: 32 - DNA, 25 bp, artificial
  synthetic oligonucleotide primer
cgatcataggcgtctcgcatatctc

(19) United States
(12) Patent Application Publication
(10) Pub. No.: US 2015/0135369 A1
(43) Pub. Date: May 14, 2015

(54) TRANSGENIC PLANTS HAVING ALTERED EXPRESSION OF PECTIN ACETYLESTERASE AND METHODS OF USING SAME

(71) Applicant: UNIVERSITY OF GEORGIA RESEARCH FOUNDATION, INC., Athens, GA (US)

(72) Inventors: Debra A. Mohnen, Athens, GA (US); Ajaya K. Biswal, Athens, GA (US)

(21) Appl. No.: 14/388,047

(22) PCT Filed: Mar. 11, 2013
(86) PCT No.: PCT/US13/30172

§ 371 Date: Sep. 25, 2014

Related U.S. Application Data
(60) Provisional application No. 61/616,570, filed on Mar. 28, 2012.

(51) Int. Cl. C12N 15/82; C12P 7/14; C12P 19/14; C12P 19/02; C12P 7/06; C12N 9/18

(52) U.S. Cl. CPC C12N 15/8246; C12P 7/06; C12N 15/8261; C12N 9/18; C12P 19/14; C12P 19/02; C12P 7/14; C12P 2203/00; C12Y 301/01006
USPC 800/298; 435/161; 435/160; 435/158; 435/136; 435/99; 435/162

(57) ABSTRACT

Provided herein are transgenic plants that include increased expression of a coding region encoding a PAE poly-peptide compared to a control plant. In one embodiment, a transgenic plant includes a phenotype of decreased recalcitrance, increased growth, or the combination thereof. Also provided herein are methods for generating transgenic plants, and methods for using transgenic plants. Examples of methods for using transgenic plants include, for instance, processing a transgenic plant described herein to result in a processed pulp, and exposing a plant material obtained from a plant described herein to conditions suitable for the production of a metabolic product.

### CROSS-REFERENCE TO RELATED APPLICATIONS

This application claims the benefit of U.S. Provisional Application Ser. No. 61/616,570, filed Mar. 28, 2012, which is incorporated by reference herein.

### GOVERNMENT FUNDING

The present invention was made with government support under Grant No. DE-AC05-00OR22725, BioEnergy Science Center (BESC), awarded by the Department of Energy. The Government has certain rights in this invention.

### BACKGROUND

Pectins are major components of the middle lamellae and primary plant cell walls in dicotyledonous species where they comprise 30-35% of cell wall dry weight. Pectin constitutes a lower percentage (2-10%) of the cell wall in species of the grass (Poaceae) family and in secondary cell walls (Mohnen, 2008, Curr. Opin. Plant Biology 11:266-277; Caffall and Mohnen, 2009, Carbohydr. Res. 344:1879-1900; Albersheim, Darvill, Roberts, Sederoff, Staehelin (2011) Plant Cell Walls, Garland Science, N.Y.; Atmodjo, Hao and Mohnen, 2013, Annu. Rev. Plant Biology 64:28.1-28.33). Pectins are highly complex polysaccharides that are rich in galacturonic acid (GalA) and include the polysaccharide domains homogalacturonan (HG), rhamnogalacturonan I (RGI), rhamnogalacturonan II (RGII) and xylogalacturonan (XGA). These pectic domains differ in both the structure of the macromolecular backbone and in the presence and diversity of side chains attached to the backbones. HG is a linear homopolymer of (1,4)-linked-α-D-galacturonic acid that can be either methylesterified at the C-6 carboxyl or carry acetyl groups at the O-2 and O-3 positions. XGA is HG with (1,3)-β-D-xylopyranoside side chains which, like HG, can be esterified by acetyl groups. RGII, which has a highly conserved structure in plants, is a complex polysaccharide with highly diverse side chains (glycosyl residues and linkages) linked to an HG backbone. Notably, RGII can be cross-linked by borate esters in cell walls. RGI differs from the structures discussed above because its backbone includes repeating [(1,2)-α-L-rhamnose-(1,4)-α-D-galacturonate]ₙ disaccharide units where n can be larger than 100. Most or all of the galacturonosyl residues in the RG-I backbone can carry acetyl groups at O-2 and O-3 and rhamnosyl residues can be substituted at O-4 by neutral sugars such as (1,4)-linked-β-D-galactans or (1,5)-linked-α-L-arabinans.

Pectins are synthesized from nucleotide sugars by at least 53 different glycosyltransferases that are located in the Golgi. The synthesized pectin is secreted into the cell wall where its structure can be significantly altered by the activity of cell wall-based enzymes. Such changes are thought to have crucial roles in multiple growth and development processes in plants. In particular HG, the main pectic component which is esterifed at O-2 and O-3 by acetyl groups and at O-6 by methyl groups during its synthesis in the medial Golgi is deposited in the cell wall in a highly esterified form. Pectin is acted upon by pectin acetylesterases (PAEs; E.C. 3.1.1.), presumably in the cell wall to result in removal of some of the pectin acetyl esters (Williamson, 1991, Phytochem. 30:445-449; Bordenave et al., 1995, Phytochem. 38:315-319).

### SUMMARY OF THE INVENTION

There is increasing interest in the generation and identification of improved lignocellulosic biomass for generation of environmentally sustainable biofuels. Lignocellulosic biomass is mostly cellulose, hemicelluloses, pectin and lignin. Pectins are the most complex polysaccharides in the plant cell wall and are enriched in the middle lamellae and primary walls in dicotyledonous species and present, albeit at lower levels, in grasses and in secondary walls. It has been proposed that pectin plays a major role in plant development and that acetylation of pectin contributes to the biological function of pectin. The acetyl esterification of the predominant pectic polysaccharide homogalacturonan (HG) occurs at positions O-2 or/and O-3 of the galacturonic acid residues as does the acetylation of the galacturonosyl residues in the RG-I backbone. As described herein, the biological effects on plant growth, development and recalcitrance of over-expression of the foxtail millet pectin acetylesterase 1 (SiPAE1) gene in rice and of the pectin acetylesterase 2 (SiPAE2) in switchgrass and rice were studied. Over-expression of SiPAE1 in rice resulted in increased growth and biomass yield and improved ethanol yields from 18-56% in diverse OE lines compared to wild type and vector control lines. On the contrary, over-expression of SiPAE2 in switchgrass resulted in a severe dwarf phenotype in seven out of ten independent events, whereas in rice two of the seven independent SiPAE2 over-expression events showed the reduced growth phenotype and three events showed increase growth. The increase in growth and ethanol yield of the rice SiPAE1 over-expression lines and the decreased growth and biomass in switchgrass and rice SiPAE2 over-expression lines indicates that acetylation of pectin significantly affects plant growth and biomass properties in rice and switchgrass, a somewhat unexpected result considering the relatively low amounts of pectin in plants, such as these grass species. The results also indicate that specific structural modifications of pectin affect plant, including grass, growth and biomass yield.

Provided herein are methods for generating a transgenic plant. In one embodiment, such a transgenic plant has decreased recalcitrance, increased growth, or the combination thereof, compared to a control plant. In one embodiment, the method includes transforming a cell of a plant with a polynucleotide to obtain a recombinant plant cell, and generating a transgenic plant from the recombinant plant cell, where the transgenic plant has increased expression of a coding region encoding a PAE polypeptide compared to a control plant. In one embodiment, the transgenic plant is a monocot plant, and in one embodiment, the transgenic plant is a dicot plant. In one embodiment, the transgenic plant is a member of the family Poaceae. In one embodiment, the member of the family Poaceae is a member of a genus Zea, Oryza, Triticum, Hordeum, Sorghum, Avena, Secale, Fagopyrum, Digitara, or Chenopodium. In one embodiment, the transgenic plant is a member of the subfamily Panicoideae. In one embodiment, the transgenic plant is a woody plant, such as a member of the genus Populus. In one embodiment, the method may further include breeding the transgenic plant with a second plant, wherein the second plant is transgenic or nontransgenic. In one embodiment, the method may further include screening the transgenic plant for an altered phenotype, such as decreased recalcitrance, increased growth, or the combination thereof.

Also provided herein is a transgenic plant that includes increased expression of a coding region encoding a PAE polypeptide compared to a control plant. In one embodiment, the transgenic plant includes a phenotype of decreased recalcitrance, increased growth, or the combination thereof. In one embodiment, the transgenic plant is a monocot plant, and in one embodiment, the transgenic plant is a dicot plant. In one embodiment, the transgenic plant is a member of the family Poaceae. In one embodiment, the member of the family Poaceae is a member of a genus Zea, Oryza, Triticum, Hordeum, Sorghum, Avena, Secale, Fagopyrum, Digitara, or Chenopodium. In one embodiment, the transgenic plant is a member of the subfamily Panicoideae. In one embodiment, the transgenic plant is a woody plant, such as a member of the genus Populus.

Further provided herein is a part of the transgenic plants described herein, wherein the part is may be a leaf, a stem, a flower, an ovary, a fruit, a seed, and a callus, or a portion thereof. Also provided herein is a progeny of a transgenic plant described herein, such as a progeny hybrid plant. Further provided is biomass obtained from the transgenic plant described herein, and a pulp of biomass obtained from a transgenic plant described herein.

Provided herein are methods for using a transgenic plant. In one embodiment, a method includes exposing a plant material obtained from a plant described herein to conditions suitable for the production of a metabolic product. In one embodiment, the exposing includes contacting the plant material with an ethanologenic microbe.

In one embodiment, a method includes processing a transgenic plant described herein to result in a processed pulp, wherein the transgenic plant includes increased expression of a coding region encoding a PAE polypeptide compared to a control plant. In one embodiment, the processing includes a pretreatment, such as a physical pretreatment, a chemical pretreatment, or a combination thereof. In one embodiment, the method further includes hydrolyzing the processed pulp. Also provided is the processed pulp.

Further provided herein are methods for producing a metabolic product. In one embodiment, a method includes contacting, under conditions suitable for the production of a metabolic product, a microbe with a composition that includes a processed pulp obtained from a transgenic plant, wherein the transgenic plant includes increased expression of a coding region encoding a PAE compared to a control plant. In one embodiment, the transgenic plant is a monocot plant, and in one embodiment, the transgenic plant is a dicot plant. In one embodiment, the transgenic plant is a member of the family Poaceae. In one embodiment, the member of the family Poaceae is a member of a genus Zea, Oryza, Triticum, Hordeum, Sorghum, Avena, Secale, Fagopyrum, Digitara, or Chenopodium. In one embodiment, the transgenic plant is a member of the subfamily Panicoideae. In one embodiment, the transgenic plant is a woody plant, such as a member of the genus Populus.

A method described herein may further include contacting a processed pulp with an ethanologenic microbe, such as a eukaryote or a prokaryote. A method described herein may further include obtaining a metabolic product. In one embodiment, a metabolic product may include an alcohol, such as, but not limited to, ethanol, butanol, a diol, or a combination thereof. In one embodiment, a metabolic product may include an organic acid, such as, but not limited to, lactic acid, acetic acid, formic acid, citric acid, oxalic acid, uric acid, or a combination thereof.

As used herein, the term “transgenic plant” refers to a plant that has been transformed to contain at least one modification to result in altered expression of a coding region. For example, a coding region in a plant may be modified to include a mutation to reduce transcription of the coding region or reduce activity of a polypeptide encoded by the coding region, or alternatively to increase transcription of the coding region or increase activity of a polypeptide encoded by the coding region. Alternatively, a plant may be transformed to include a polynucleotide that interferes with expression of a coding region. For example, a plant may be modified to express an antisense RNA or a double stranded RNA that silences or reduces expression of a coding region by decreasing translation of an mRNA encoded by the coding region. In some embodiments more than one coding region may be affected. The term “transgenic plant” includes whole plants, plant parts (stems, roots, leaves, fruit, etc.) or organs, plant cells, seeds, and progeny of same. A transformed plant of the current invention can be a direct transfectant, meaning that the DNA construct was introduced directly into the plant, such as through Agrobacterium, or the plant can be the progeny of a transfected plant. The second or subsequent generation plant can be produced by sexual reproduction, i.e., fertilization. Furthermore, the plant can be a gametophyte (haploid stage) or a sporophyte (diploid stage). A transgenic plant may have a phenotype that is different from a plant that has not been transformed.

As used herein, the terms “coding region” and “coding sequence” are used interchangeably and refer to a nucleotide sequence that encodes a polypeptide and, when placed under the control of appropriate regulatory sequences expresses the encoded polypeptide. The boundaries of a coding region are generally determined by a translation start codon at its 5′ end and a translation stop codon at its 3′ end. A “regulatory sequence” is a nucleotide sequence that regulates expression of a coding sequence to which it is operably linked. Non-limiting examples of regulatory sequences include promoters, enhancers, transcription initiation sites, translation start sites, translation stop sites, and transcription terminators. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

As used herein, the term “wild-type” refers to a plant cell, seed, plant component, plant tissue, plant organ or whole plant that has not been genetically modified or treated in an experimental sense.

As used herein, the term “control plant” refers to a plant that is the same species as a transgenic plant, but has not been transformed with the same polynucleotide used to make the transgenic plant.

As used herein, the term “plant tissue” encompasses any portion of a plant, including plant parts (stems, branches, roots, leaves, fruit, seed mucilage and root mucilage etc.) or organs, plant cells, and seeds. Plant cells include suspension cultures, callus, embryos, meristematic regions, callus tissue, leaves, roots, shoots, gametophytes, sporophytes, pollen, seeds, and microspores. Plant tissues can be grown in liquid or solid culture, or in soil or suitable media in pots, greenhouses or fields. As used herein, “plant tissue” also refers to a clone of a plant, seed, progeny, or propagule, whether generated sexually or asexually, and descendents of any of these, such as cuttings or seeds.

Unless indicated otherwise, as used herein, “altered expression of a coding region” refers to a change in the transcription of a coding region, a change in translation of an mRNA encoded by a coding region, or a change in the activity of a polypeptide encoded by the coding region.

As used herein, “transformation” refers to a process by which a polynucleotide is inserted into the genome of a plant cell. Such an insertion includes stable introduction into the plant cell and transmission to progeny. Transformation also refers to transient insertion of a polynucleotide, wherein the resulting transformant transiently expresses a polypeptide that may be encoded by the polynucleotide.

As used herein, “phenotype” refers to a distinguishing feature or characteristic of a plant which can be altered as described herein by modifying expression of at least one coding region in at least one cell of a plant. The modified expression of at least one coding region can confer a change in the phenotype of a transformed plant by modifying any one or more of a number of genetic, molecular, biochemical, physiological, morphological, or agronomic characteristics or properties of the transformed plant cell or plant as a whole. Whether a phenotype of a transgenic plant is altered is determined by comparing the transformed plant with a plant of the same species that has not been transformed with the same polynucleotide (a “control plant”).

As used herein, “mutation” as used herein refers to a modification of the natural nucleotide sequence of a coding region or an operably linked regulatory region in such a way that the polypeptide encoded by the modified nucleic acid is altered structurally and/or functionally, or the coding region is expressed at an altered level. Mutations may include, but are not limited to, mutations in a 5′ or 3′ untranslated region (UTR) or an exon, and such mutations may be a deletion, insertion, or point mutation to result in, for instance, a codon encoding a different amino acid or a stop to translation.

As used herein, the term “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e.g., dimers, tetramers). Thus, the terms peptide, oligopeptide, and protein are all included within the definition of polypeptide and these terms are used interchangeably.

As used herein, a polypeptide may be “structurally similar” to a reference polypeptide if the amino acid sequence of the polypeptide possesses a specified amount of sequence similarity and/or sequence identity compared to the reference polypeptide. Thus, a polypeptide may be “structurally similar” to a reference polypeptide if, compared to the reference polypeptide, it possesses a sufficient level of amino acid sequence identity, amino acid sequence similarity, or a combination thereof.

As used herein, the term “polynucleotide” refers to a polymeric form of nucleotides of any length, either ribonucleotides, deoxynucleotides, peptide nucleic acids, or a combination thereof, and includes both single-stranded molecules and double-stranded duplexes. A polynucleotide can be obtained directly from a natural source, or can be prepared with the aid of recombinant, enzymatic, or chemical techniques. A polynucleotide described herein may be isolated. An “isolated” polynucleotide is one that has been removed from its natural environment. Polynucleotides that are produced by recombinant, enzymatic, or chemical techniques are considered to be isolated and purified by definition, since they were never present in a natural environment.

As used herein, a polynucleotide may have “sequence similarity” to a reference polynucleotide if the nucleotide sequence of the polynucleotide possesses a specified amount of sequence sequence identity compared to a reference polynucleotide. Thus, a polynucleotide may have “structural similarity” to a reference polynucleotide if, compared to the reference polynucleotide, it possesses a sufficient level of nucleotide sequence identity.

An “isolated” polynucleotide or polypeptide is one that has been removed from its natural environment. Polynucleotides and polypeptides that are produced by recombinant, enzymatic, or chemical techniques are considered to be isolated and purified by definition, since they were never present in a natural environment.

A “regulatory sequence” is a nucleotide sequence that regulates expression of a coding sequence to which it is operably linked. Nonlimiting examples of regulatory sequences include promoters, enhancers, transcription initiation sites, translation start sites, translation stop sites, transcription terminators, and poly(A) signals. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

The term “complementary” refers to the ability of two single stranded polynucleotides to base pair with each other, where an adenine on one polynucleotide will base pair to a thymine or uracil on a second polynucleotide and a cytosine on one polynucleotide will base pair to a guanine on a second polynucleotide.

“Hybridization” includes any process by which a strand of a nucleic acid sequence joins with a second nucleic acid sequence strand through base-pairing. Thus, strictly speaking, the term refers to the ability of a target sequence to bind to a test sequence, or vice-versa.

“Hybridization conditions” are typically classified by degree of “stringency” of the conditions under which hybridization is measured. The degree of stringency can be based, for example, on the calculated (estimated) melting temperature (Tm) of the nucleic acid sequence binding complex or probe. Calculation of Tm is known in the art (see Sambrook et al., 2001, Molecular Cloning, A Laboratory Manual, 3d Ed., Cold Spring Harbor Laboratory Press, Cold Spring Harbor, N.Y.). For example, “maximum stringency” typically occurs at about Tm −5° C. (5° below the Tm of the probe); “high stringency” at about 5-10° C. below the Tm; “intermediate stringency” at about 10-20° C. below the Tm of the probe; and “low stringency” at about 20-25° C. below the Tm. In general, hybridization conditions are carried out under high ionic strength conditions, for example, using 6×SSC or 6×SSPE. Under high stringency conditions, hybridization is followed by two washes with low salt solution, for example 0.5×SSC, at the calculated temperature. Under medium stringency conditions, hybridization is followed by two washes with medium salt solution, for example 2×SSC. Under low stringency conditions, hybridization is followed by two washes with high salt solution, for example 6×SSC. Functionally, maximum stringency conditions may be used to identify nucleic acid sequences having strict identity or near-strict identity with the hybridization probe; while high stringency conditions are used to identify nucleic acid sequences having about 80% or more sequence identity with the probe. For applications requiring high selectivity, one will typically desire to employ relatively stringent conditions to form the hybrids, e.g., one will select relatively high temperature conditions. Hybridization conditions, including moderate stringency and high stringency, are provided in Sambrook et al., Molecular Cloning: A Laboratory Manual, Second Edition, Cold Spring Harbor Press (1989); Sambrook et al., Molecular Cloning, A Laboratory Manual, 3d Ed., Cold Spring Harbor Laboratory Press, Cold Spring Harbor, N.Y. (2001).

As used herein, “recalcitrance” refers to the natural resistance of plant cell walls to microbial and/or enzymatic and/or chemical deconstruction (see Fu et al., 2011, Proc. Natl. Acad. Sci. USA 108:3803-3808).

Conditions that are “suitable” for an event to occur, or “suitable” conditions are conditions that do not prevent such events from occurring. Thus, these conditions permit, enhance, facilitate, and/or are conducive to the event.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Genomic PCR of independent events of SiPAE1-OE plants in rice. (A) List of independent transformation events of SiPAE1-OE rice lines. Each number represents a line from the same event and each small letter a-d represents multiple clonal lines for that event. For example two plant lines were obtained from event P1-6, plants P1-6a and P1-6b while four plant lines from the same event were obtained for Pa-65 (plants P1-65 a-d). (B) Primers used for genomic PCR.

FIG. 2. SiPAE1-OE lines in rice. (A) Transgenic rice over-expression lines of foxtail millet gene PAE1 (SiPAE1). WT, wild type; 6A-17B, vector control; and P1-series; PAE1-OE lines. (B) Relative gene expression of SiPAE1 in WT, Control and several SiPAE1-OE rice lines measured by qRT-PCR and compared to actin.

FIG. 3. Plant height and number of tillers in wild type, control and SiPAE1-OE rice lines. Statistical analysis was performed using Statistica 5.0 and with one-way analysis of variance (ANOVA) followed by Tukey's multiple comparison test. Significance P values are expressed as *P<0.05 and **P<0.001.

FIG. 4. Dry weight of wild type, control and SiPAE1-OE rice lines. Statistical analysis was performed using Statistica 5.0 and with one-way analysis of variance (ANOVA) followed by Tukey's multiple comparison test. Significance P values are expressed as *P<0.05 and **P<0.001.

FIG. 5. Monosaccharide composition analysis of wild type, control and SiPAE1-OE rice lines. Statistical analysis was performed using Statistica 5.0 and with one-way analysis of variance (ANOVA) followed by Tukey's multiple comparison test. Significance P values are expressed as *P<0.05.

FIG. 6. Bioconversion of rice SiPAE1-OE lines to ethanol. Ethanol yield of rice SiPAE1-OE plants following fermentation after pretreatment at 180° C. for 7.5 min with 0.5% H₂SO₄ in comparison to wild type and vector controls 6A-14A and 6A-17B. Data are average +/−S.D. of two biological replicates with two technical replicates per biological sample.

FIG. 7. Weight loss of rice SiPAE1-OE biomass during fermentation. (A) Time course of biomass weight of wild type, vector control (6A-17B) and rice SiPAE1-OE transgenics over time during fermentation bottle. B. Weight of wild type, vector control (6A-17B) and rice SiPAE1-OE transgenics at the end of fermentation. Data are average +/−S.D. of two biological replicates with two technical replicates per biological sample.

FIG. 8. Genomic PCR of independent events of rice SiPAE2-OE plants. (A) List of independent events of rice SiPAE2-OE lines. (B) Primers used for genomic PCR.

FIG. 9. Rice SiPAE2-OE lines. Transgenic rice over-expressing foxtail millet PAE2 (SiPAE2). WT, wild type; 6A-17B, vector control; and P2-series; PAE2-OE lines.

FIG. 10. Plant height and number of tillers in wild type, vector control and SiPAE2-OE rice lines. Statistical analysis was performed using Statistica 5.0 and with one-way analysis of variance (ANOVA) followed by Tukey's multiple comparison test. Significance P values are expressed as *P<0.05 and **P<0.001.

FIG. 11. Dry weight biomass of wild type, control and SiPAE2-OE rice lines. Statistical analysis was performed using Statistica 5.0 and with one-way analysis of variance (ANOVA) followed by Tukey's multiple comparison test. Significance P values are expressed as *P<0.05 and **P<0.001.

FIG. 12. Transgenic switchgrass overexpressing foxtail millet PAE2. Plants 1-9 transgenic lines, TC—transgenic control. Transgenic lines 1-7 showed a severe dwarf phenotype with very short internodes and thicker leaves compared to control lines.

FIG. 13. Genomic PCR of independent events of transgenic switchgrass plants overexpressing foxtail millet PAE2 gene (lanes 1-10). Transgenic lines 1-10 were positive for the transgene. The transgenic line was negative for the gene but positive for RFP and HPT.

FIG. 14. Relative gene expression of SiPAE2 transgenic and control switchgrass plants. More than a 60-fold increase in transgene expression compared to the endogenous gene (PvPAE2) was detected in some lines. The native PvPAE2 had varied relative expression across the lines. SiPAE2 expression was lower in lines 8 and 9 than the native gene expression.

FIG. 15. Nucleotide sequence of PAE1 used for transformation of switchgrass (SEQ ID NO:6).

FIG. 16. Relative gene expression of SiPAE1 transgenic and control switchgrass plants. More than a 60-fold increase in transgene expression in PAE1-1 and PAE1-2 was detected in comparison to wild type (ST2). Similarly more than a 30-fold increase in transgene expression in PAE1-3, PAE1-4 and PAE1-5 was observed in comparison to wild type (ST1).

FIG. 17. Glucose (A) and total sugar (B) release from control and SiPAE1 overexpression switchgrass lines. Significance P values are expressed as *P<0.05 by statistical analysis using Statistica 5.0 with one-way analysis of variance (ANOVA) followed by Tukey's multiple comparison test.

FIG. 18. Example of a nucleotide sequence (SEQ ID NO:1) encoding a PAE1 polypeptide (SEQ ID NO:2). The PAE1 polypeptide includes the signal peptide sequence MAPRRRRAWPAAAAVVTAVVTAAA (residues 1-24 of SEQ ID NO:2).

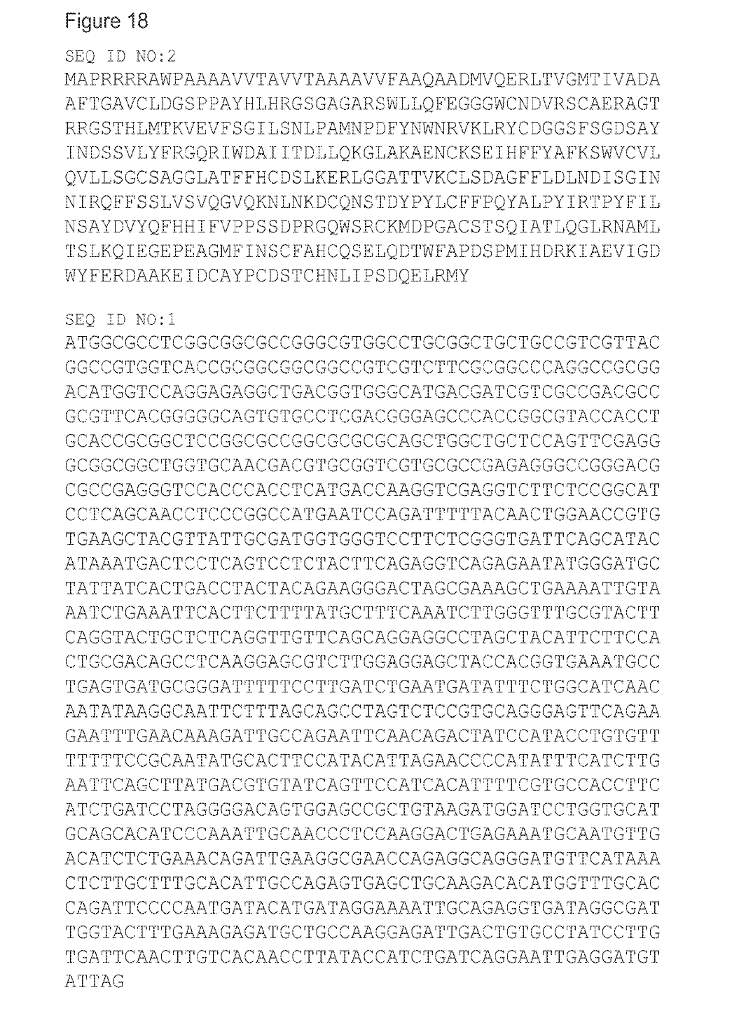

FIG. 19. Alignment of PAE polypeptides from Setaria italica L. (SiPAE1, SEQ ID NO:2), Populus trichocarpa (PtPAE1, SEQ ID NO:3), and Vigna radiata var. radiata (VrPAE, SEQ ID NO:4) using the ClustalW algorithm.

### DETAILED DESCRIPTION OF ILLUSTRATIVE EMBODIMENTS

Provided herein are cultured plant cells and plants that include alterations in expression of polypeptides having pectin acetylesterase (PAE) activity. The cultured plant cells and plants may be transgenic or may be natural variants. A polypeptide having PAE activity is referred to herein as a PAE polypeptide. The alterations in expression of a PAE polypeptide may include, but are not limited to, an increase in expression of an active PAE polypeptide, expression of an inactive PAE polypeptide, expression of an active PAE polypeptide, an increase in PAE activity, a decrease in expression of an active PAE polypeptide, expression of an inactive PAE polypeptide, expression of a PAE polypeptide that is altered to have decreased activity, the absence of detectable expression of a PAE polypeptide, or a decrease in PAE activity. More than one polypeptide may be altered in a cell or plant. In one embodiment, such modifications may be achieved by mutagenesis of a coding sequence encoding a PAE polypeptide.

PAE polypeptides catalyse the specific deacetylation of esterified homogalacturonan and rhamnogalacturonan within plant cell walls, a process which may increase or decrease pectin solubility in water and allow homogalacturonan and rhamnogalacturonan in the plant cell wall to become a target for pectin-degrading or modifying enzymes such as pectate lyase and rhamnogalacturonan hydrolase, since these enzymes do not, generally, cleave acetylated homogalacturonan or rhamnogalacturonan. As used herein, a polypeptide having PAE activity means a polypeptide that catalyzes, under suitable conditions, the removal of pectin acetyl esters from a substrate. An example of a substrate is the pectic polysaccharide homogalacturonan that has acetyl groups at the O-2 and/or the O-3 position of the HG α-D-galacturonic acids (Williamson, 1991, Phytochem. 30:445-449, Bordenave et al., 1995, Phytochem. 38:315-319). Another example is rhamnogalacturonan-I (RG-I) that has acetyl groups at the O-2 and/or the O-3 of the α-D-galacturonosyl residues in the RG-I backbone (Albersheim, Darvill, Roberts, Sederoff, Staehelin (2011) Plant Cell Walls, Garland Science, N.Y.). Such substrates are available using cell wall fractionation, purification and modification methods known to those familiar in the state of the art in the field. For example, HG acetylated oligomers (degree of acetylation [DA] 25%) and RG-I acetylated oligomers (DA 120%) are readily available using methods known to a person skilled in the art. In one embodiment, PAE activity refers to the ability of a PAE polypeptide to remove an acetyl groups from the O-2 positions of α-D-galacturonic acids present in an HG molecule. In one embodiment, PAE activity refers to the ability of a PAE polypeptide to remove acetyl groups from the O-3 positions of α-D-galacturonic acids present in an HG molecule. In one embodiment, PAE activity refers to the ability of a PAE polypeptide to remove acetyl groups from the O-2 and the O-3 positions of α-D-galacturonic acids present in an HG molecule. In one embodiment, PAE activity refers to the ability of a PAE polypeptide to remove acetyl groups from the O-2 and the O-3 positions of α-D-galacturonic acids present in the backbone of RG-I.

Methods for determining whether a polypeptide has the ability to remove pectin acetyl esters from a substrate are known in the art. Whether a polypeptide has PAE activity may be determined by in vitro assays. In one embodiment, an in vitro assay that evaluates a candidate polypeptide's ability to remove acetyl groups from the O-2 and/or the O-3 positions of α-D-galacturonic acids may be carried out as follows. Briefly, 3 micrograms polypeptide (for instance, recombinant polypeptide) is incubated with 15 milligrams/ml pectin in 100 mM Tris HCL buffer, pH 7.0 at 35° C. for 30 minutes. The pectin substrate may, for example, be acetylated HG with different differing degrees of acetylation such as, but not limited to, 20, 50, or 100. The pectin substrate may also be acetylated RG-1 with differing degrees of acetylation such as, but not limited to, 50 or 100. The pectin may also be present in a proteoglycan such as arabinoxylan-pectin-arabinogalactan protein 1 (APAP1) (Tan et al., 2013, Plant Cell, 25:270-287) and as such the substrate may also include, for example, regions of acetylated xylan. Acetate released from the reaction can be quantified by using a Acetate Kinase Format Kit (K-ACETAK) from Megazyme (Megazyme International Ireland) or with 79464 HPLC Columns (Hamilton Company; PRP-X300 Ion Exclusion Columns; 150 mm×4.1 mm (L×I.D.); 7 micron particle size).

Whether a polypeptide has PAE activity may be determined by in vivo assays. In one embodiment, PAE activity may be measured from cell walls, for example, from leaves or tillers from wild type or PAE over-expressing switchgrass lines using methods such as the following. Leaf tissue (5 gm) can be ground in 10 mL buffer such as 50 mM sodium succinate buffer (pH 5) containing 1 M NaCl to isolate a supernatant fraction enriched in PAE removed from the cell walls by the salt in the buffer and the supernatant collected by centrifugation at 10,000 g for 10 min at 4° C. Following de-salting by dialysis using, for example, 3.5 kDa cutoff dialysis tubing against several changes of 50 mM succinate buffer, pH 5, the protein content in the supernatant may be measured by, for example, the Bradford assay. PAE enzyme activity can be measured in an assay mixture (1 mL) containing 0.1 M Na-K-Pi buffer, pH 7, 2 mM pNPA pNPA (p-nitrophenyl acetate) and protein (40 μg protein/mL). Hydrolysis of pNPA may be measured spectrophotometrically at 405 nm by the formation of p-nitrophenol and release of acetate may also be quantified using Acetate Kinase Format Kit (K-ACETAK) from Megazyme (Megazyme International Ireland) or with 79464 HPLC Columns (Hamilton Company; PRP-X300 Ion Exclusion Columns; 150 mm×4.1 mm (L×I.D.); 7 micron particle size).

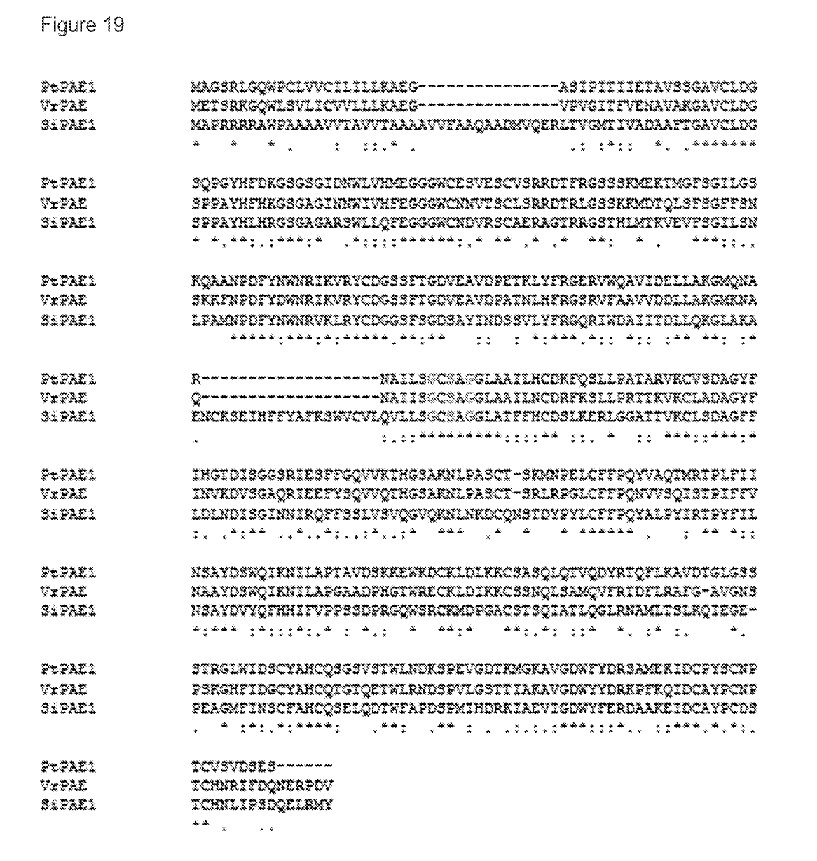

Examples of PAE polypeptides from Setaria italica L., Populus trichocarpa, and Vigna radiata var. radiata, are shown in FIG. 19 (SEQ ID NO:2, 3, and 4, respectively). Other plants have homologs, including orthologs and paralogs, of these PAE polypeptides. Other examples of PAE polypeptides include polypeptides having structural similarity with a reference polypeptide selected from SEQ ID NO:2, SEQ ID NO:3, or SEQ ID NO:4

In one embodiment, a PAE polypeptide includes a signal sequence at the amino-terminus. In one embodiment, a PAE polypeptide does not include a signal sequence. Methods for predicting and determining the signal sequence of a polypeptide are known and routine. The signal sequence of the PAE polypeptide depicted at SEQ ID NO:2 is residues 1-24, MAPRRRRAWPAAAAVVTAVVTAAA. As a skilled person will appreciate, an active PAE polypeptide expressed as a transgene in a plant cell, including a transgenic plant, will include amino acids that act as a signal sequence. Likewise, expression of an active PAE polypeptide in vitro, for instance for use in an in vitro assay of activity, may or may not include amino acids that act as a signal sequence. Unless noted otherwise or apparent from the context in which it is discussed, reference to a PAE polypeptide refers to a PAE polypeptide that includes a signal sequence; however, the signal sequence need not be residues 1-24 of SEQ ID NO:2.

Structural similarity of two polypeptides can be determined by aligning the residues of the two polypeptides (for example, a candidate polypeptide and any appropriate reference polypeptide described herein) to optimize the number of identical amino acids along the lengths of their sequences; gaps in either or both sequences are permitted in making the alignment in order to optimize the number of identical amino acids, although the amino acids in each sequence must nonetheless remain in their proper order. In one embodiment a reference polypeptide is a polypeptide described herein, such as SEQ ID NO:2, or amino acids 25 - 434 of SEQ ID NO:2. A candidate polypeptide is the polypeptide being compared to the reference polypeptide. A candidate polypeptide can be isolated, for example, from a plant, or can be produced using recombinant techniques, or chemically or enzymatically synthesized.

Unless modified as otherwise described herein, a pair-wise comparison analysis of amino acid sequences can be carried out using the Blastp program of the blastp suite-2sequences search algorithm, as described by Tatiana et al., (FEMS Microbiol Lett, 174, 247-250 (1999)), and available on the National Center for Biotechnology Information (NCBI) website. The default values for all blastp suite-2sequences search parameters may be used, including general paramters: expect threshold=10, word size=3, short queries=on; scoring parameters: matrix=BLOSUM62, gap costs=existence:11 extension:1, compositional adjustments=conditional compositional score matrix adjustment. Alternatively, polypeptides may be compared using the BESTFIT algorithm in the GCG package (version 10.2, Madison Wis.).

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a polypeptide disclosed herein may be selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH2. Likewise, a polypeptide containing deletions or additions of one or more contiguous or noncontiguous amino acids that do not eliminate PAE activity of the polypeptide are also contemplated.

PAE polypeptides are members of the Carbohydrate-Active enZYmes (CAZy) carbohydrate esterase family 13 (CE13) (Cantarel et al., 2009, Nucleic Acids Res 37:D233-238). The CAZy database describes the families of structurally-related catalytic and/or carbohydrate-binding modules (or functional domains) of enzymes that degrade, modify, or create glycosidic bonds (Cantarel et al., 2009, Nucleic Acids Res., 37:D233-238; Campbell et al., 1997, Biochem. J. 326:929-939; Coutinho et al., 2003, J. Mol. Biol. 328:307-317).

A PAE polypeptide typically includes conserved amino acids and conserved domains. FIG. 19 depicts an amino acid alignment of three PAE polypeptides. In FIG. 19, an asterisk (*) marks the location of identical residues, a colon (:) marks the location of conserved residues, and a period (.) marks the location of weakly conserved residues. A PAE polypeptide includes a conserved GXSXG motif (SEQ ID NO:5, where X is any amino acid), which is a characteristic of the Serine hydrolase superfamily.

Thus, as used herein, reference to an amino acid sequence of SEQ ID NO:2, or amino acids 25-434 of SEQ ID NO:2, can include a polypeptide with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to the reference amino acid sequence.

Alternatively, as used herein, reference to an amino acid sequence of SEQ ID NO:2, or amino acids 25-434 of SEQ ID NO:2, can include a polypeptide with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference amino acid sequence.

Examples of polynucleotides encoding SEQ ID NO:2 are shown at SEQ ID NO:1. It should be understood that a polynucleotide encoding a PAE polypeptide is not limited to a nucleotide sequence disclosed herein, but also includes the class of polynucleotides encoding the PAE polypeptide as a result of the degeneracy of the genetic code. For example, the nucleotide sequence SEQ ID NO:1 is but one member of the class of nucleotide sequences encoding a polypeptide having the amino acid sequence SEQ ID NO:2. The class of nucleotide sequences encoding a selected polypeptide sequence is large but finite, and the nucleotide sequence of each member of the class may be readily determined by one skilled in the art by reference to the standard genetic code, wherein different nucleotide triplets (codons) are known to encode the same amino acid.

While the polynucleotide sequences described herein are listed as DNA sequences, it is understood that the complements, reverse sequences, and reverse complements of the DNA sequences can be easily determined by the skilled person. It is also understood that the sequences disclosed herein as DNA sequences can be converted from a DNA sequence to an RNA sequence by replacing each thymidine nucleotide with a uridine nucleotide.

Also provided herein are polynucleotide sequences having sequence similarity with, in one embodiment, SEQ ID NO:1 and encoding a PAE polypeptide, and in another embodiment, nucleotides 74-1,305 of SEQ ID NO:1 and encoding a PAE polypeptide. Sequence similarity of two polynucleotides can be determined by aligning the residues of the two polynucleotides (for example, a candidate polynucleotide and any appropriate reference polynucleotide described herein) to optimize the number of identical nucleotides along the lengths of their sequences; gaps in either or both sequences are permitted in making the alignment in order to optimize the number of identical nucleotides, although the nucleotides in each sequence must nonetheless remain in their proper order. A reference polynucleotide may be a polynucleotide described herein. A candidate polynucleotide is the polynucleotide being compared to the reference polynucleotide. A candidate polynucleotide may be isolated, for example, from a plant, or can be produced using recombinant techniques, or chemically or enzymatically synthesized. A candidate polynucleotide may be present in the genome of a plant and predicted to encode a PAE polypeptide.

A pair-wise comparison analysis of nucleotide sequences can be carried out using the Blastn program of the BLAST search algorithm, available through the World Wide Web, for instance at the internet site maintained by the National Center for Biotechnology Information, National Institutes of Health. Preferably, the default values for all Blastn search parameters are used. Alternatively, sequence similarity may be determined, for example, using sequence techniques such as GCG FastA (Genetics Computer Group, Madison, Wis.), MacVector 4.5 (Kodak/IBI software package) or other suitable sequencing programs or methods known in the art.

Thus, as used herein, a candidate polynucleotide useful in the methods described herein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% nucleotide sequence identity to a reference nucleotide sequence.

Also provided herein are polynucleotides capable of hybridizing to a nucleotide sequence encoding a PAE polypeptide. In one embodiment, the polynucleotide may be SEQ ID NO:1, or a complement thereof, and encoding a PAE polypeptide. In one embodiment, the polynucleotide may be the nucleotides 74-1,305 of SEQ ID NO:1 encoding amino acids 25 -434 of SEQ ID NO:2, or a complement thereof, and encoding a PAE polypeptide. The hybridization conditions may be medium to high stringency. A maximum stringency hybridization can be used to identify or detect identical or near-identical polynucleotide sequences, while an intermediate or low stringency hybridization can be used to identify or detect polynucleotide sequence homologs.

Provided herein are methods of using PAE polypeptides and polynucleotides encoding PAE polypeptides. In one embodiment, methods include altering expression of plant PAE coding regions for purposes including, but not limited to (i) investigating function of pectin acetylation on plant growth and development porocesses, (ii) investigating mechanisms of pectin acetylation, (iii) effecting a change in plant phenotype, and (iv) using plants having an altered phenotype.

In one embodiment, methods include altering expression of a PAE coding region present in the genome of a plant. The plant may be a wild-type plant. In one embodiment, an additional PAE coding region is present in the genome of a plant. In one embodiment a wild-type plant is a member of the family Poaceae.

Techniques which can be used in accordance with methods to alter expression of a PAE coding region, include, but are not limited to: (i) over-expression the coding region; (ii) disrupting a coding region's transcript, such as disrupting a coding region's mRNA transcript, (iii) disrupting the activity of a polypeptide encoded by a coding region, (iv) disrupting the coding region itself, or (v) modifying the timing of expression of the coding region by placing it under the control of a non-native promoter. The use of antisense RNAs, ribozymes, double-stranded RNA interference (dsRNAi), gene knockouts, and gene knockins are valuable techniques for discovering the functional effects of a coding region and for generating plants with a phenotype that is different from a wild-type plant of the same species.

Over-expression of a coding region may be accomplished by cloning the coding region into an expression vector and introducing the vector into recipient cells. Alternatively, over-expression can be accomplished by introducing exogenous promoters into cells to drive expression of coding regions residing in the genome. The effect of over-expression of a given coding region on the phenotype of a plant can be evaluated by comparing plants over-expressing the coding region to control plants.

Methods for increasing expression of a coding region are routinely used in the art and include, for example, over-expression driven by appropriate promoters, the use of transcription enhancers, or the use of translation enhancers. Regulatory elements, such as promoters or enhancer elements, may be introduced in an appropriate position (typically upstream) of a coding region present in the genome of a plant to upregulate expression of the coding region. For example, endogenous promoters may be altered in vivo by mutation, deletion, and/or substitution (see, Kmiec, U.S. Pat. No. 5,565,350; Zarling et al., WO 93/22443), or promoters may be introduced into a plant cell in the proper orientation and distance from a coding region encoding a polypeptide disclosed herein so as to control the expression of the gene.

If polypeptide expression is desired, it is generally desirable to include a polyadenylation region at the 3′-end of a polynucleotide coding region. The polyadenylation region can be derived from the natural coding region, from a variety of other plant coding regions, or from T-DNA. The 3′ end sequence to be added may be derived from, for example, the nopaline synthase or octopine synthase coding regions, or alternatively from another plant coding region, or from another eukaryotic coding region.

An intron sequence may also be added to the 5′ untranslated region (UTR) or the coding sequence of a partial coding sequence to increase the amount of the mature message that accumulates in the cytosol. Inclusion of a spliceable intron in the transcription unit in both plant and animal expression constructs has been shown to increase expression at both the mRNA and protein levels up to 1000-fold (Buchman and Berg, 1988, Mol. Cell biol. 8: 4395-4405; Callis et al., 1987, Genes Dev 1:1183-1200). Such intron enhancement of gene expression is typically greatest when placed near the 5′ end of the transcription unit. Use of the maize introns Adh1-S intron 1, 2, and 6, the Bronze-1 intron are known in the art (see, e.g., The Maize Handbook, Chapter 116, Freeling and Walbot, Eds., Springer, N.Y. (1994).

Another method for increasing expression of a coding region includes T-DNA activation tagging (Hayashi et al. Science (1992) 1350-1353), which involves insertion of T-DNA, usually containing a promoter, a translation enhancer, and/or an intron, in the genomic region of the codign region of interest or 10 kb up- or downstream of the coding region in a configuration such that the promoter directs expression of the targeted coding region. Typically, regulation of expression of the targeted coding region by its natural promoter is disrupted and the coding region is controlled by the newly introduced promoter. The promoter is typically embedded in a T-DNA. This T-DNA is randomly inserted into the plant genome, for example, through Agrobacterium infection and leads to modified expression of coding regions near the inserted T-DNA. The resulting transgenic plants show dominant phenotypes due to modified expression of genes close to the introduced promoter.

Antisense RNA, ribozyme, and dsRNAi technologies typically target RNA transcripts of coding regions, usually mRNA. Antisense RNA technology involves expressing in, or introducing into, a cell an RNA molecule (or RNA derivative) that is complementary to, or antisense to, sequences found in a particular mRNA in a cell. By associating with the mRNA, the antisense RNA can inhibit translation of the encoded gene product. The use of antisense technology to reduce or inhibit the expression of specific plant genes has been described, for example in European Patent Publication No. 271988, Smith et al., 1988, Nature, 334:724-726, and Smith et. al., 1990, Plant Mol. Biol., 14:369-379.

A ribozyme is an RNA that has both a catalytic domain and a sequence that is complementary to a particular mRNA. The ribozyme functions by associating with the mRNA (through the complementary domain of the ribozyme) and then cleaving the message using the catalytic domain.

RNA interference (RNAi) involves a post-transcriptional gene silencing (PTGS) regulatory process, in which the steady-state level of a specific mRNA is reduced by sequence-specific degradation of the transcribed, usually fully processed mRNA without an alteration in the rate of de novo transcription of the target gene itself The RNAi technique is discussed, for example, in Small, 2007, Curr. Opin. Biotechnol., 18:148-153; McGinnis, 1010, Brief. Funct. Genomics, 9(2): 111-117.

Disruption of a coding region may be accomplished by T-DNA based inactivation. For instance, a T-DNA may be positioned within a polynucleotide coding region described herein, thereby disrupting expression of the encoded transcript and protein. T-DNA based inactivation can be used to introduce into a plant cell a mutation that alters expression of the coding region, e.g., decreases expression of a coding region or decreases activity of the polypeptide encoded by the coding region. For instance, mutations in a coding region and/or an operably linked regulatory region may be made by deleting, substituting, or adding a nucleotide(s).The use of T-DNA based inactiviation is discussed, for example, in Azpiroz-Leehan et al. (1997, Trends in Genetics, 13:152-156). Disruption of a coding region may also be accomplished using methods that include transposons, homologous recombination, and the like.

Altering expression of a PAE coding region may be accomplished by using a portion of a polynucleotide described herein. In one embodiment, a polynucleotide for altering expression of a PAE coding region in a plant cell includes one strand, referred to herein as the sense strand, of at least 19 nucleotides, for instance, 19, 20, 21, 22, 23, 24, 25, 26, 27, 28, or 29 nucleotides (e.g., lengths useful for dsRNAi and/or antisense RNA). In one embodiment, a polynucleotide for altering expression of a PAE coding region in a plant cell includes substantially all of a coding region, or in some cases, an entire coding region (e.g., lengths useful for T-DNA based inactivation). The sense strand is substantially identical, preferably, identical, to a target coding region or a target mRNA. As used herein, the term “identical” means the nucleotide sequence of the sense strand has the same nucleotide sequence as a portion of the target coding region or the target mRNA. As used herein, the term “substantially identical” means the sequence of the sense strand differs from the sequence of a target mRNA at least 1%, 2%, 3%, 4%, or 5% of the nucleotides, and the remaining nucleotides are identical to the sequence of the mRNA.

In one embodiment, a polynucleotide for altering expression of a PAE coding region in a plant cell includes one strand, referred to herein as the antisense strand. The antisense strand may be at least 19 nucleotides, for instance, 19, 20, 21, 22, 23, 24, 25, 26, 27, 28, or 29 nucleotides. In one embodiment, a polynucleotide for altering expression of a PAE coding region in a plant cell includes substantially all of a coding region, or in some cases, an entire coding region. An antisense strand is substantially complementary, preferably, complementary, to a target coding region or a target mRNA. As used herein, the term “substantially complementary” means that at least 1%, 2%, 3%, 4%, or 5% of the nucleotides of the antisense strand are not complementary to a nucleotide sequence of a target coding region or a target mRNA.

Methods are readily available to aid in the choice of a series of nucleotides from a polynucleotide described herein. For instance, algorithms are available that permit selection of nucleotides that will function as dsRNAi and antisense RNA for use in altering expression of a coding region. The selection of nucleotides that can be used to selectively target a coding region for T-DNA based inactivation may be aided by knowledge of the nucleotide sequence of the target coding region.

Polynucleotides described herein, including nucleotide sequences which are a portion of a coding region described herein, may be operably linked to a regulatory sequence. An example of a regulatory region is a promoter. A promoter is a nucleic acid, such as DNA, that binds RNA polymerase and/or other transcription regulatory elements. A promoter facilitates or controls the transcription of DNA or RNA to generate an RNA molecule from a nucleic acid molecule that is operably linked to the promoter. The RNA can encode a PAE polypeptide, an antisense RNA molecule, or a molecule useful in RNAi. Promoters useful in the invention include constitutive promoters, inducible promoters, and/or tissue preferred promoters for expression of a polynucleotide in a particular tissue or intracellular environment, examples of which are known to one of ordinary skill in the art.

A constitutive promoter refers to a promoter that is transcriptionally active during most, but not necessarily all, phases of growth and development and under most environmental conditions, in at least one cell, tissue or organ. Examples of useful constitutive plant promoters include, but are not limited to, the cauliflower mosaic virus (CaMV) 35S promoter, (Odel et al., 1985, Nature, 313:810), the nopaline synthase promoter (An et al., 1988, Plant Physiol., 88:547), and the octopine synthase promoter (Fromm et al., 1989, Plant Cell 1: 977).

An inducible promoter has induced or increased transcription initiation in response to a chemical (for a review see Gatz, 1997, Annu Rev. Plant Physiol. Plant Mol. Biol., 48:89-108), environmental, or physical stimulus. Examples of inducible promoters include, but are not limited to, auxin-inducible promoters (Baumann et al., 1999, Plant Cell, 11:323-334), cytokinin-inducible promoters (Guevara-Garcia, 1998, Plant Mol. Biol., 38:743-753), and gibberellin-responsive promoters (Shi et al., 1998, Plant Mol. Biol., 38:1053-1060). Additionally, promoters responsive to heat, light, wounding, pathogen resistance, and chemicals such as methyl jasmonate or salicylic acid, can be used, as can tissue or cell-type specific promoters such as xylem-specific promoters (Lu et al., 2003, Plant Growth Regulation 41:279-286).

A tissue preferred promoter is one that is capable of preferentially initiating transcription in certain organs or tissues, such as the leaves, roots, seed tissue etc. For example, a root-specific promoter is a promoter that is transcriptionally active predominantly in plant roots, substantially to the exclusion of any other parts of a plant, while still allowing for any leaky expression in these other plant parts. Promoters able to initiate transcription in certain cells only are referred to herein as cell-specific.

A seed-specific promoter is transcriptionally active predominantly in seed tissue, but not necessarily exclusively in seed tissue (in cases of leaky expression). The seed-specific promoter may be active during seed development and/or during germination. The seed specific promoter may be endosperm/aleurone/embryo specific. Examples of seed-specific promoters (endosperm/aleurone/embryo specific) are described in Russinova and Reuzeau (US Patent Application 20120331584). Further examples of seed-specific promoters are given in Qing Qu and Takaiwa (2004, Plant Biotechnol. J., 2:113-125).

A green tissue-specific promoter is a promoter that is transcriptionally active predominantly in green tissue, substantially to the exclusion of any other parts of a plant, while still allowing for any leaky expression in these other plant parts.

Another example of a tissue-specific promoter is a meristem-specific promoter, which is transcriptionally active predominantly in meristematic tissue, substantially to the exclusion of any other parts of a plant, while still allowing for any leaky expression in these other plant parts.

Another example of a regulatory region is a transcription terminator. Suitable transcription terminators are known in the art and include, for instance, a stretch of 5 consecutive thymidine nucleotides.

Thus, in one embodiment, a polynucleotide that is operably linked to a regulatory sequence may be in a “sense” orientation, where transcription produces an mRNA that encodes a PAE polypeptide. In one embodiment, a polynucleotide that is operably linked to a regulatory sequence may be in an “antisense” orientation, the transcription of which produces a polynucleotide which can form secondary structures that affect expression of a target coding region in a plant cell. In another embodiment, the polynucleotide that is operably linked to a regulatory sequence may yield one or both strands of a double-stranded RNA product that initiates RNA interference of a target coding region in a plant cell.

A polynucleotide may be present in a vector. A vector is a replicating polynucleotide, such as a plasmid, phage, or cosmid, to which another polynucleotide may be attached so as to bring about the replication of the attached polynucleotide. Construction of vectors containing a polynucleotide of the invention employs standard ligation techniques known in the art. See, e.g., Sambrook et al, Molecular Cloning: A Laboratory Manual.,

Cold Spring Harbor Laboratory Press (1989). A vector can provide for further cloning (amplification of the polynucleotide), i.e., a cloning vector, or for expression of the polynucleotide, i.e., an expression vector. The term vector includes, but is not limited to, plasmid vectors, viral vectors, cosmid vectors, transposon vectors, and artificial chromosome vectors. A vector may result in integration into a cell's genomic DNA. A vector may be capable of replication in a bacterial host, for instance E. coli or Agrobacterium tumefaciens. Preferably the vector is a plasmid. In some embodiments, such as those related to making a dsRNA, a polynucleotide can be present in a vector as two separate complementary polynucleotides, each of which can be expressed to yield a sense and an antisense strand of a dsRNA, or as a single polynucleotide containing a sense strand, an intervening spacer region, and an antisense strand, which can be expressed to yield an RNA polynucleotide having a sense and an antisense strand of the dsRNA.

Selection of a vector depends upon a variety of desired characteristics in the resulting construct, such as a selection marker, vector replication rate, and the like. Suitable host cells for cloning or expressing the vectors herein are prokaryotic or eukaryotic cells. Suitable eukaryotic cells include plant cells. Suitable prokaryotic cells include eubacteria, such as gram-negative organisms, for example, E. coli or A. tumefaciens.

A selection marker is useful in identifying and selecting transformed plant cells or plants. Examples of such markers include, but are not limited to, a neomycin phosphotransferase (NPTII) gene (Potrykus et al., 1985, Mol. Gen. Genet., 199:183-188), which confers kanamycin resistance, a hygromycin B phosphotransfease (HPTII) gene (Kaster, et al, 1983, Nuc. Acid. Res. 19: 6895-6911), and a bialaphos acetyltransferase (bar) gene, conferring resistance to bialaphos (Richards et al., 2001, Plant Cell Rep. 20, 48-54, and Somleva et al., 2002, Crop Sci. 42, 2080-2087). Cells expressing the NPTII gene can be selected using an appropriate antibiotic such as kanamycin or G418. The HPTII gene encodes a hygromycin-B 4-O-kinase that confers hygromycin B resistance. Cells expressing HPTII gene can be selected using the antibiotic of hygromycin B (Kaster, et al, 1983, Nuc. Acid. Res. 19: 6895-6911, Blochlinger and Diggelmann, 1984, Mol. Cell. Biol. 4 (12): 2929-2931). Other commonly used selectable markers include a mutant EPSP synthase gene (Hinchee et al., 1988, Bio/Technology 6:915-922), which confers glyphosate resistance; and a mutant acetolactate synthase gene (ALS), which confers imidazolinone or sulphonylurea resistance (Conner and Santino, 1985, European Patent Application 154,204).

Polynucleotides described herein can be produced in vitro or in vivo. For instance, methods for in vitro synthesis include, but are not limited to, chemical synthesis with a conventional DNA/RNA synthesizer. Commercial suppliers of synthetic polynucleotides and reagents for in vitro synthesis are well known. Methods for in vitro synthesis also include, for instance, in vitro transcription using a circular or linear expression vector in a cell free system. Expression vectors can also be used to produce a polynucleotide described herein in a cell, and the polynucleotide may then be isolated from the cell.

The invention also provides host cells having altered expression of a coding region described herein. In one embodiment, the expression is increased. As used herein, a host cell includes the cell into which a polynucleotide described herein was introduced, and its progeny, which may or may not include the polynucleotide. Accordingly, a host cell can be an individual cell, a cell culture, or cells that are part of an organism. The host cell can also be a portion of an embryo, endosperm, sperm or egg cell, or a fertilized egg. In one embodiment, the host cell is a plant cell.

Provided herein are transgenic plants having altered expression of a coding region. In one embodiment, the expression is increased. In one embodiment, a transgenic plant may be homozygous or heterozygous for a modification that results in altered expression of a coding region.

In one embodiment, a host cell or a transgenic plant may have an increase in expression of a PAE polypeptide. A host cell or a transgenic plant having increased PAE expression may have an increased amount of mRNA encoding a PAE polypeptide, may have an increased amount of PAE polypeptide, may have increased PAE activity, or a combination thereof, compared to a control plant. The increase in expression of a PAE polypeptide may be increased by at least 1%, at least 5%, at least 10%, at least 20%, at least 30%, at least 40%, at least 50%, at least 60%, at least 70%, at least 80%, or at least 90% compared to the expression of a PAE polypeptide in a control plant.

Also provided herein are natural variants of plants. In one embodiment, a natural variant has increased expression of a PAE polypeptide, where the change in PAE expression is relative to the level of expression of the PAE polypeptide in a natural population of the same species of plant. Natural populations include natural variants, and at a low level, extreme variants (Studer et al., 2011, Proc. Nat. Acad. Sci., USA, 108:6300-6305). The level of expression of PAE polypeptide in an extreme variant may vary from the average level of expression of the PAE polypeptide in a natural population by at least 5%, at least 10%, at least 15%, at least 20%, or at least 25%. The average level of expression of the PAE polypeptide in a natural population may be determined by using at least 50 randomly chosen plants of the same species as the putative extreme variant.

A plant may be an angiosperm or a gymnosperm. The polynucleotides described herein may be used to transform a variety of plants, both monocotyledonous (e.g grasses, sugar cane, corn, grains, oat, wheat, barley, rice, and the like), dicotyledonous (e.g., Arabidopsis, Brassica, tobacco, potato, tomato, peppers, melons, legumes, alfalfa, oaks, eucalyptus, maple, poplar, aspen, cottonwood, and the like).

The plants also include switchgrass (Panicum virgatum), millet (including foxtail millet), turfgrass, sugar beet, lettuce, carrot, strawberry, cassava, sweet potato, geranium, soybean, and various types of woody plants.

In one embodiment, a plant is a member of the family Poaceae. For instance, a plant may be a member of a genus selected from Zea, Oryza, Triticum, Hordeum, Sorghum, Avena, Secale, Fagopyrum, Digitara, and Chenopodium. For instance, a plant may be a member of the subfamily Panicoideae. In one embodiment, the plant is a grass, such as switchgrass, rice, barley, wheat, bamboo, millet (including foxtail millet), sorghum, maize (corn stover), and micanthus.

In one embodiment, the plants are woody plants, which are trees or shrubs whose stems live for a number of years and increase in diameter each year by the addition of woody tissue. Woody plants include trees such as palm oak, pine, maple, fir, apple, fig, plum acacia, aspen, and willow. Woody plants also include rose and grape vines, including Vitus spp. Plants of significance in the commercial biomass industry and useful in the methods disclosed herein include, but are not limited to, members of the family Salicaceae, such as Populus spp. (e.g., Populus trichocarpa, Populus deltoides), members of the family Pinaceae, such as Pinus spp. (e.g., Pinus taeda [Loblolly Pine]), and Eucalyptus spp.

Also provided is the plant material (such as, for instance, stems, branches, roots, leaves, fruit, etc.) derived from a plant described herein. A plant material derived from a plant described herein may include cell wall material that includes a decreased amount of acetyl groups at the O-2 and/or O-3 positions of galacturonosyl residues in homogalacturonan and/or rhamnogalacturonan. In one embodiment, the plant material is present in a plant material-derived product such as lumber (including, for instance, dimensional lumber and engineered lumber), paper products, plant biomass-derived biomaterials, and agricultural products. In one embodiment, a plant material-derived product is a pulp. As used herein, “pulp” refers to a mechanically, chemically and/or biologically processed wood or non-wood plant material that contains cell wall material. Cell wall material includes cell walls, cell-wall polymers and/or molecules (such as oligosaccharides) that are derived from cell wall polymers. Cell wall polymers include cellulose, hemicellulose, pectin and/or lignin. Processing to generate a pulp may increase the susceptibility of the cell wall polysaccharides to hydrolysis and fermentation. Examples of pulp include, for instance, woodchips and sawdust. Also provided is pulp derived from a plant and/or plant material described herein. In one embodiment, the plant material is agricultural waste.

Transformation of a plant with a polynucleotide described herein to result in altered PAE polypeptide expression, for instance, increased PAE expression, may yield a change in one or more phenotypes compared to a control plant. The phenotypes include, but are not limited to, increased growth (such as increased height and/or increased number of tillers), increased biomass, altered monosaccharide composition (such as increased glucose, decreased xylose, and/or increased total sugar), alterd bioproduct production (such as increased ethanol yield and/or decreased time for production of ethanol), and changes in cell wall composition. Changes in cell wall include changes in cell wall polysaccharide content and/or changes in pectin acetylation, such as acetylation of HG or RG-I. In one embodiment a phenotype is an increased plant height compared to a control plant. In one embodiment, a phenotype is an increased number of tillers. In one embodiment, a phenotype is increased biomass compared to a control plant. In one embodiment, a phenotype is increased glucose content compared to a control plant. In one embodiment, a phenotype is increased ethanol yield compared to a control plant.

In one embodiment a phenotype is reduced recalcitrance compared to a control plant. Methods for measuring recalcitrance are routine and include, but are not limited to, measuring changes in the extractability of carbohydrates, where an increase in extractability suggests a cell wall that is more easily solubilized, and thus, decreased recalcitrance. Another test for measuring changes in recalcitrance uses microbes as described in Mohnen et al. (WO 2011/130666). In one embodiment, the recalcitrance of a plant or plant part described herein is reduced by at least 2-fold, at least 3-fold, at least 4-fold, at least 5-fold, or at least 10-fold compared to a control plant or plant part.

Other phenotypes present in a transgenic plant described herein may include yielding biomass with reduced recalcitrance and from which sugars can be released more efficiently for use in biofuel and biomaterial production, yielding biomass which is more easily deconstructed and allows more efficient use of wall structural polymers and components, and yielding biomass that will be less costly to refine for recovery of sugars and biomaterials.

Phenotype can be assessed by any suitable means. The biochemical characteristics of lignin, cellulose, carbohydrates such as pectin, and other plant extracts can be evaluated by standard analytical methods including spectrophotometry, fluorescence spectroscopy, HPLC, mass spectroscopy, molecular beam mass spectroscopy, near infrared spectroscopy, nuclear magnetic resonance spectroscopy, and tissue staining methods.

One method that can be used to evaluate the phenotype of a transgenic plant is glycome profiling. Glycome profiling gives information about the presence of carbohydrate structures in plant cell walls, including changes in the extractability of carbohydrates, such as xylose, from cell walls (Zhu et al., 2010, Mol. Plant, 3:818-833; Pattathil et al., 2010, Plant Physiol., 153:514-525), the latter providing information about larger scale changes in wall structure. Diverse plant glycan-directed monoclonal antibodies are available from, for instance, CarboSource Services (Athens, Ga.), and PlantProbes (Leeds, UK). The change in extractability may be an increase or a decrease of one or more carbohydrates in an extracted fraction compared to a control plant. In one embodiment the change is an increase of one or more carbohydrates in an extracted fraction compared to a control plant. Examples of solvents useful for evaluating the extractability of carbohydrates include, but are not limited to, oxalate, carbonate, KOH, and chlorite.

Transgenic plants described herein may be produced using routine methods. Methods for transformation and regeneration are known to the skilled person. Transformation of a plant cell with a polynucleotide described herein may be achieved by any known method for the insertion of nucleic acid sequences into a prokaryotic or eukaryotic host cell, including Agrobacterium-mediated transformation protocols, viral infection, whiskers, electroporation, microinjection, polyethylene glycol-treatment, heat shock, lipofection, particle bombardment, and chloroplast transformation.

Transformation techniques for dicotyledons are known in the art and include Agrobacterium-based techniques and techniques that do not require Agrobacterium. Non-Agrobacterium techniques involve the uptake of exogenous genetic material directly by protoplasts or cells. This may be accomplished by, for instance, PEG or electroporation mediated-uptake, particle bombardment-mediated delivery, or microinjection. In each case the transformed cells may be regenerated to whole plants using standard techniques known in the art.

Techniques for the transformation of monocotyledon species include, but are not limited to, direct gene transfer into protoplasts using PEG or electroporation techniques, particle bombardment into callus tissue or organized structures, as well as Agrobacterium-mediated transformation.

The cells that have been transformed may be grown into plants in accordance with conventional techniques. See, for example, McCormick et al. (1986, Plant Cell Reports, 5:81-84). These plants may then be grown and evaluated for expression of desired phenotypic characteristics. These plants may be either pollinated with the same transformed strain or different strains, and the resulting hybrid having desired phenotypic characteristics identified. Two or more generations may be grown to ensure that the desired phenotypic characteristics are stably maintained and inherited and then seeds harvested to ensure stability of the desired phenotypic characteristics have been achieved.

Provided herein are methods for using a plant and/or plant material described herein. In one embodiment, a method includes using a plant and/or plant material. In one embodiment, a plant and/or plant material may be used to produce a plant material-derived product. Examples of plant material-derived products include lumber and pulp. Plant material-derived products may be used in, for instance, furniture making, construction, foods (including fruit and vegetable juices). Plant material-derived products, such as pulp, may be used as a food additive, a liquid absorbent, as animal bedding, and in gardening. Plants and/or plant material described herein may also be used as a feedstock for livestock. Plants with reduced recalcitrance are expected to be more easily digested by an animal and more efficiently converted into animal mass. Accordingly, in one embodiment, a method includes using a plant and/or plant material described herein as a source for a feedstock, and includes a feedstock that has plant material from a transgenic plant as one of its components.

In one embodiment, a method includes producing a metabolic product. A process for producing a metabolic product from a transgenic plant described herein may include processing a plant (also referred to as pretreatment of a plant), enzymatic hydrolysis, fermentation, and/or recovery of the metabolic product. Each of these steps may be practiced separately, thus included herein are methods for processing a transgenic plant to result in a pulp, methods for hydrolyzing a pulp that contain cells from a transgenic plant, and methods for producing a metabolic product from a pulp.

There are numerous methods or combinations of methods known in the art and routinely used to process plants. The result of processing a plant is a pulp. Plant material, which can be any part of a plant, may be processed by any means, including, for instance, mechanical, chemical, biological, or a combination thereof. Mechanical pretreatment breaks down the size of plant material. Biomass from agricultural residues is often mechanically broken up during harvesting. Other types of mechanical processing include milling or aqueous/steam processing. Chipping or grinding may be used to typically produce particles between 0.2 and 30 mm in size. Methods used for plant materials may include intense physical pretreatments such as steam explosion and other such treatments (Peterson et al., U.S. Patent Application 20090093028). Common chemical pretreatment methods used for plant materials include, but are not limited to, dilute acid, alkaline, organic solvent, ammonia, sulfur dioxide, carbon dioxide or other chemicals to make the biomass more available to enzymes. Biological pretreatments are sometimes used in combination with chemical treatments to solubilize lignin in order to make cell wall polysaccharides more accessible to hydrolysis and fermentation. In one embodiment, a method for using transgenic plants described herein includes processing plant material to result in a pulp. In one embodiment, transgenic plants described herein, such as those with reduced recalcitrance, are expected to require less processing than a control plant. In some embodiments, the conditions described below for different types of processing are expected to result in greater amounts of carbohydrate oligomers and carbohydrate monomers when used with a plant described herein compared to a control plant.

Steam explosion is a common method for pretreatment of plant biomass and increases the amount of cellulose available for enzymatic hydrolysis (Foody, U.S. Pat. No. 4,461,648). Generally, the material is treated with high-pressure saturated steam and the pressure is rapidly reduced, causing the materials to undergo an explosive decompression. Steam explosion is typically initiated at a temperature of 160-260° C. for several seconds to several minutes at pressures of up to 4.5 to 5 MPa. The biomass is then exposed to atmospheric pressure. The process typically causes degradation of cell wall complex carbohydrates and lignin transformation. Addition of H₂SO₄, SO₂, or CO₂ to the steam explosion reaction can improve subsequent cellulose hydrolysis (Morjanoff and Gray, 1987, Biotechnol. Bioeng. 29:733-741).

In ammonia fiber explosion (AFEX) pretreatment, biomass is treated with approximately 1-2 kg ammonia per kg dry biomass for approximately 30 minutes at pressures of 1.5 to 2 MPa. (Dale, U.S. Pat. No. 4,600,590; Dale, U.S. Pat. No. 5,037,663; Mes-Hartree, et al. 1988, Appl. Microbiol. Biotechnol., 29:462-468). Like steam explosion, the pressure is then rapidly reduced to atmospheric levels, boiling the ammonia and exploding the lignocellulosic material. AFEX pretreatment appears to be especially effective for biomass with a relatively low lignin content, but not for biomass with high lignin content such as newspaper or aspen chips (Sun and Cheng, 2002, Bioresource Technol., 83:1-11).

Concentrated or dilute acids may also be used for pretreatment of plant biomass. H₂SO₄ and HCl have been used at high concentrations, for instance, greater than 70%. In addition to pretreatment, concentrated acid may also be used for hydrolysis of cellulose (Hester et al., U.S. Pat. No. 5,972,118). Dilute acids can be used at either high (>160° C.) or low (<160° C.) temperatures, although high temperature is preferred for cellulose hydrolysis (Sun and Cheng, 2002, Bioresource Technol., 83:1-11). H₂SO₄ and HCl at concentrations of 0.3 to 2% (wt/wt) and treatment times ranging from minutes to 2 hours or longer can be used for dilute acid pretreatment.

Hot water can also be used as a pretreatment of plant biomass (Studer et al, 2011, Proc. Natl. Acad. Sci., U.S.A., 108:6300-6305). In one embodiment, hydrothermal treatment is at a temperature between 130° C. and 200° C., such as 140° C., 160, or 180° C., and for a time between 5 minutes and 120 minutes. In one embodiment, examples of times include at least 5 minutes, at least 10 minutes, at least 20 minutes, at least 30 minutes, at least 45 minutes, or at least 60 minutes. In one embodiment, examples of times include no greater than 120 minutes, no greater than 105 minutes, no greater than 90 minutes, or no greater than 75 minutes. The temperature and time used depends upon the source and condition of the biomass used, and an effective combination of time and temperature can be easily determined by the skilled person. In one embodiment, the biomass is exposed to a hydrothermal pretreatment having a severity level of logR0 between 2 and 5, where severity is defined as R0=t*exp ((T-100)/14.73) with t the time in minutes and T the temperature in degree Celsius (Lloyd and Wyman, 2005, Bioresource Technology, 96(18):1967-1977; Overend and Chornet, 1987, Phil. Trans. R. Soc. Lond. (A321), 523-536; and Wyman and Kumar, US Published Patent Application 20110201084). Examples of severity levels include at least 2, at least 2.5, at least 3, at least 3.5, at least 4, at least 4.5, and at least 5.

Other pretreatments include alkaline hydrolysis (Qian et al., 2006, Appl. Biochem. Biotechnol., 134:273; Galbe and Zacchi, 2002, Appl. Microbiol. Biotechnol., 59:618), oxidative delignification, organosolv process (Pan et al., 2005, Biotechnol. Bioeng., 90:473; Pan et al., 2006, Biotechnol. Bioeng., 94:851; Pan et al., 2006, J. Agric. Food Chem., 54:5806; Pan et al., 2007, Appl. Biochem. Biotechnol., 137-140:367), or biological pretreatment.

Methods for hydrolyzing a pulp may include enzymatic hydrolysis. Enzymatic hydrolysis of processed biomass may include the use of cellulases. Some of the pretreatment processes described above include hydrolysis of complex carbohydrates, such as hemicellulose and cellulose, to monomer sugars. Others, such as organosolv, prepare the substrates so that they will be susceptible to hydrolysis. This hydrolysis step can in fact be part of the fermentation process if some methods, such as simultaneous saccharification and fermentation (SSF), are used. Otherwise, the pretreatment may be followed by enzymatic hydrolysis with cellulases.

A cellulase may be any enzyme involved in the degradation of the complex carbohydrates in plant cell walls to fermentable sugars, such as glucose, xylose, mannose, galactose, and arabinose. The cellulolytic enzyme may be a multicomponent enzyme preparation, e.g., cellulase, a monocomponent enzyme preparation, e.g., endoglucanase, cellobiohydrolase, glucohydrolase, beta-glucosidase, or a combination of multicomponent and monocomponent enzymes. The cellulolytic enzymes may have activity, e.g., hydrolyze cellulose, either in the acid, neutral, or alkaline pH-range.

A cellulase may be of fungal or bacterial origin, which may be obtainable or isolated from microorganisms which are known to be capable of producing cellulolytic enzymes. Useful cellulases may be produced by fermentation of the above-noted microbial strains on a nutrient medium containing suitable carbon and nitrogen sources and inorganic salts, using procedures known in the art.

Examples of cellulases suitable for use in the present invention include, but are not limited to, CELLUCLAST (available from Novozymes A/S) and NOVOZYME (available from Novozymes A/S). Other commercially available preparations including cellulase which may be used include CELLUZYME, CEREFLO and ULTRAFLO (Novozymes A/S), LAMINEX and SPEZYME CP (Genencor Int.), and ROHAMENT 7069 W (Rohm GmbH).

The steps following pretreatment, e.g., hydrolysis and fermentation, can be performed separately or simultaneously. Conventional methods used to process the plant material in accordance with the methods disclosed herein are well understood to those skilled in the art. Detailed discussion of methods and protocols for the production of ethanol from biomass are reviewed in Wyman (1999, Annu Rev. Energy Environ., 24:189-226), Gong et al. (1999, Adv. Biochem. Eng. Biotech., 65: 207-241), Sun and Cheng (2002, Bioresource Technol., 83:1-11), and Olsson and Hahn-Hagerdal (1996, Enzyme and Microb. Technol., 18:312-331). The methods of the present invention may be implemented using any conventional biomass processing apparatus (also referred to herein as a bioreactor) configured to operate in accordance with the invention. Such an apparatus may include a batch-stirred reactor, a continuous flow stirred reactor with ultrafiltration, a continuous plug-flow column reactor (Gusakov, A. V., and Sinitsyn, A. P., 1985, Enz. Microb. Technol., 7: 346-352), an attrition reactor (Ryu, S. K., and Lee, J. M., 1983, Biotechnol. Bioeng., 25: 53-65), or a reactor with intensive stirring induced by an electromagnetic field (Gusakov, A. V., Sinitsyn, A. P., Davydkin, I. Y., Davydkin, V. Y., Protas, O. V., 1996, Appl. Biochem. Biotechnol., 56: 141-153). Smaller scale fermentations may be conducted using, for instance, a flask.

The conventional methods include, but are not limited to, saccharification, fermentation, separate hydrolysis and fermentation (SHF), simultaneous saccharification and fermentation (SSF), simultaneous saccharification and cofermentation (SSCF), hybrid hydrolysis and fermentation (HHF), and direct microbial conversion (DMC). The fermentation can be carried out by batch fermentation or by fed-batch fermentation.

SHF uses separate process steps to first enzymatically hydrolyze plant material to glucose and then ferment glucose to ethanol. In SSF, the enzymatic hydrolysis of plant material and the fermentation of glucose to ethanol are combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF includes the coferementation of multiple sugars (Sheehan, J., and Himmel, M., 1999, Enzymes, energy and the environment: A strategic perspective on the U.S. Department of Energy's research and development activities for bioethanol, Biotechnol. Prog., 15: 817-827). HHF includes two separate steps carried out in the same reactor but at different temperatures, i.e., high temperature enzymatic saccharification followed by SSF at a lower temperature that the fermentation strain can tolerate. DMC combines all three processes (cellulase production, cellulose hydrolysis, and fermentation) in one step (Lynd, L. R., Weimer, P. J., van Zyl, W. H., and Pretorius, I. S., 2002, Microbiol. Mol. Biol. Reviews, 66: 506-577).

The final step may be recovery of the metabolic product. Examples of metabolic products include, but are not limited to, alcohols, such as ethanol, butanol, a diol, and organic acids such as lactic acid, acetic acid, formic acid, citric acid, oxalic acid, and uric acid. The method depends upon the metabolic product that is to be recovered, and methods for recovering metabolic products resulting from microbial fermentation of plant material are known to the skilled person and used routinely. For instance, when the metabolic product is ethanol, the ethanol may be distilled using conventional methods. For example, after fermentation the metabolic product, e.g., ethanol, may be separated from the fermented slurry. The slurry may be distilled to extract the ethanol, or the ethanol may be extracted from the fermented slurry by micro or membrane filtration techniques. Alternatively the fermentation product may be recovered by stripping.

The present invention is illustrated by the following examples. It is to be understood that the particular examples, materials, amounts, and procedures are to be interpreted broadly in accordance with the scope and spirit of the invention as set forth herein.

### EXAMPLE 1

Foxtail millet pectin acetylesterase 1 (SiPAE1) was over-expressed in rice. An increase in plant height, increase in the number of tillers and a higher biomass yield in SiPAE1 over-expression transgenic rice lines was observed. The SiPAE1 over-expression rice lines gave increased ethanol yields by up to 18-56% (depending upon the line) in comparison to wild type and control lines based on biomass fermentation analyses. Foxtail millet pectin acetylesterase 2 (SiPAE2) was also over-expressed in switchgrass and rice. SiPAE2 over-expression switchgrass lines had severely reducted growth in switchgrass while SiPAE2 over-expression rice plants displayed less dramatic effects. The results show that pectin acetylation has a significant positive effect on plant growth and ethanol production in rice and also affects switchgrass growth and development. Furthermore, the results show that diverse PAEs have unique effects on plant growth and sugar release, and thus, that PAEs offer unique tools for improving biomass as biofeedstock for biofuel and biomaterial production.

### Materials and Methods

### Gene Isolation, Cloning and Transformation of SiPAE 1 and SiPAE2 in Rice.

The foxtail millet (Setaria italica L.) pectin acetylesterasel (SiPAE1) gene was isolated by screening of a foxtail millet fosmid library. The isolated genomic sequence of the SiPAE1 and SiPAE2 gene was cloned into a pCR8 entry vector and the gene was then cloned into a pANIC 6A vector by GATEWAY recombination (Mann et al., 2012, Plant Biotechnol J. 10: 226-236) for overexpression via biolistic transformation into rice. The pANIC 6A vector contained a DsRed-type red fluorescent protein (RFP) reporter gene, pporRFP, isolated from the coral Porites porites (Alieva et al., 2008, PLoS ONE 3:e2680) and the hph gene in the Gateway® cassette under control of the OsAct1 promoter (Mann et al., 2012, Plant Biotechnol J. 10: 226-236). The SiPAE1 and SiPAE2 genes were cloned into pANIC-6A as per the manufacturer's instructions (Invitrogen, Carlsbad, Calif., USA). Rice events were identified using real-time tracking of pporRFP expression which was monitored via in vivo fluorescence microcopy using an RFP filter. RFP expression was observed using an Olympus MVX10 microscope equipped with an RFP filter cube, model U-XL49004 from Olympus. Callus tissue was excited with 545/25 nm light and visualized using a 605/70 nm emissions filter. Three month old rice callus cultures were used for transient expression assays for stable transformations of rice according to Mann et al. 2011, BMC Biotechnology, 11:74). Prior to being moved to the greenhouse, root samples were collected and GUS-staining was performed on all the transgenic plants. An untransformed control was regenerated without selection. Prior to harvesting tissue for GUS staining the trangenics were allowed to grow in the greenhouse for approximately two months (Mann et al. 2011, BMC Biotechnology, 11:74).

### Genomic PCR of SiPAE1 and SiPAE2 Over-Expressed Rice Lines.

DNA isolation for PCR was performed on ground young tissues as per Lassner et al. (1989, Plant Mol Biol Rep 7:116-128) and the DNA was quantified using an Eppendorf BioPhotometer (Hamburg, Germany) and standardized to a concentration of 30 ng ml⁻¹ by dilution with Type I water. Standard PCR was performed using the GoTaq™ kit by Promega and 1-μl of DNA template at a concentration of 30 ng ul⁻¹ or 1 pg ul⁻¹ of plasmid DNA. PCR conditions were: (1) 94° C. 4 minutes, (2) 94° C. 30 seconds (3) Tm X° C. 30 seconds, (4) 72° C. 1 minute per kb, steps (2-4) repeated for 32 cycles, (5) 72° C. 7 minutes. To detect the PAE1 gene in rice lines, primers from PAE1 front end and back end were used for amplification of the DNA. The PAE1 lines [nine independent events with eighteen lines, P1-6 (a,b); P1-22 (a,b); P1-51a; P1-56 (a,b); P1-59a; P1-60a; P1-61 (a-d); P1-64a; P1-65 (a-d)] were analyzed by amplification using the ZmUbi 1900F forward primer 5′ TTTAGCCCTGCCTTCATACG (SEQ ID NO:7) and PAE1-R reverse primer 5′ CAAGACAGTTTACGCTCGATCA (SEQ ID NO:8) to give a 767 by product for PAE1 front end and PAE1-F forward primer 5′ AACTGGAACCGTGTGAAGCT (SEQ ID NO:9) and OCS-R reverse primer 5′ CAACGTGCACAACAGAATTGA (SEQ ID NO:10) to give a 704 by product for PAE1 back end. The wild type rice lines were analyzed by amplification using the Os_rbcS F forward primer 5′ CTTGGTGAGCTGCAGAGATGG (SEQ ID NO:11) and Os_rbcS F reverse primer 5′ AGGGTCTCGAACTTCTTGATG (SEQ ID NO:12) to give a 288 by product. Similarly for PAE2 rice lines [seven independent events with thirteen lines, P2-3; P2-7 (a,b); P2-10 (a,b); P2-13; P2-15 (a,b); P2-16 (a,b); P2-21 (a-c)] were analyzed by amplification using the ZmUbi 1900F forward primer 5′ TTTAGCCCTGCCTTCATACG (SEQ ID NO:13) and PAE2-R reverse primer 5′ GGATTCCAGAGAAGGCAATCT (SEQ ID NO:14) to give a 654 by product for PAE2 front end and PAE2-F forward primer 5′ ACCTCCAGAGGGTTGTTCAT (SEQ ID NO:15) and OCS-R reverse primer 5′ CAACGTGCACAACAGAATTGA (SEQ ID NO:16) to give a 678 by product for PAE2 back end. Non-transgenic template DNA, water, and the transformation plasmid were used as PCR controls. The vector control 6A lines (eight independent events with thirteen lines, 6A-4 (a, b, c); 6A-6; 6A-8b; 6A-11; 6A-14 (a, b); 6A-17b; 6A-18 (a, b); 6A-19 (a,b) were analyzed using the Hyg117 forward primer 5′ CGATGTAGGAGGGCGTGGATA (SEQ ID NO:17) and Hyg938 reverse primer 5′ CTTCTGCGGGCGATTTGTG (SEQ ID NO:18) to give a 822 by product. PCR products were visualized on a 0.5×TBE 1% agarose gel supplemented with 0.5 μg ml⁻¹ ethidium bromide (data not shown).

### Plant Material and Growth Conditions.

Taipei 309 rice seeds were provided by the USDA National Plant Germplasm System and maintained and cultured as previously described (Mann et al., 2011, BMC Biotechnology, 11:74). After transfer to soil, rice lines overexpressing SiPAE1 and SiPAE2 were grown in greenhouse at 25° C. with 60% constant relative humidity and a photoperiod 16/8 light/dark cycle.

### Quantitative Real-Time PCR of SiPAE1 Over-Expression Rice Lines.

For expression analysis, leaves from independent transgenic rice lines were harvested and frozen immediately in liquid nitrogen and stored at −80° C. until use. All the tissues were ground to a fine powder using N² in a chilled mortar and pestle. Total RNA was extracted using TRIREAGENT followed by DNAse (Promega) treatment to remove genomic DNA contamination. First strand cDNA synthesis was performed using 2 μg of total RNA with a blend of oligo (dT) and random primers in the Hi capacity cDNA Synthesis Kit (Applied Biosystems, USA) according to the manufacturer's instructions. The primers used to amplify the SiPAE1 transcripts of the above tissue were as follows: SiPAE1 (forward, 5′-GATGTCCGATGTTTGTGTGC (SEQ ID NO:19); reverse, 5′-CTCGACCTTGGTCATGAGGT (SEQ ID NO:20). For an internal standard two primers for rice actin were used for quantification, OsActin (forward, 5′-CTTCATAGGAATGGAAGCTGCGGGTA (SEQ ID NO:21); reverse, 5′-CGACCACCTTGATCTTCATGCTGCTA (SEQ ID NO:22)). PCR reactions were performed in a 96-well plate with AB 7900HT Fast Real-Time PCR System. Detection of products was by binding of the fluorescent DNA dye SYBR Green (AB Power SYBR Green PCR mastermix) to the PCR products. All assays were carried out in triplicate and one-set of no-template controls was included per gene amplification. A PCR reaction contained a total volume of 25 μl with appropriate cDNA, SYBR Green, and both forward and reverse primers. Thermal cycling conditions were as follows: initial activation step 10 minutes at 95° C., followed by 15 seconds at 95° C., 30 seconds at 55° C., 30 seconds at 72° C. for 45 cycles, 1 minute 95° C., 1 minute 55° C., a melting curve program (80 cycles, 10 s each of 0.5° C. elevations starting at 55° C.) and a cooling step to 4° C. The presence of one product per gene was confirmed by analysis of the disassociation curves. SDS software v2.3 (Applied Biosystems, USA) was used to calculate the first significant fluorescence signal above noise, the threshold cycle (Ct). Relative quantification was performed using the standard curve method and transcript accumulation of each gene was normalized to the quantity of expressed rice actin gene.

### Monossacharide Composition Analysis of SiPAE1 in Rice.

Rice biomass samples were analyzed for carbohydrate composition using a quantitative saccharification assay ASTM E 1758-01 (ASTM 2003) and HPLC method NREL/TP 51-42623. The resulting neutralized samples were analyzed for carbohydrate composition using high performance liquid chromatography (HPLC) La chrom elite system (Hitachi High Technologies America, Inc.) equipped with a refractive index detector (model L-2490) and UV-VIS detector (model L-2420). The carbohydrates (glucose, xylose, galactose, mannose, and arabinose) were separated using an Aminex HPX-87P column (Bio-Rad Laboratories, Inc.) with a 0.6 mL/min flow rate of water and a column temperature of 80° C. (Fu et al., 2011, Proc Natl Acad Sci U S A. 108:3803-3808).

### Enzyme Hydrolysis and Fermentation of SiPAE 1 in Rice.

Ethanol fermentation of rice biomass was conducted in sealed 70 mL reusable BBL Septi-Chek bottles using 1 g rice biomass per bottle in 20 mL. S. cerevisiae D5A(ATCC 200062) was grown in YEPD medium (Difco, Detroit, Mich.) as inoculum. Fermentations contained 1% yeast extract, 50 mM citric acid buffer pH 4.8, enzyme, 0.5 mL cells from ˜24 hr old inoculum, 50 ug/mL streptomycin, biomass and water to 20 mL. (Mielenz et al., 2009, Bioresour Technol 100:3532-3539). Fermentations were conducted with shaking @36° C. using a New Brunswick C24 shaker (New Brunswick Instrument Company, New Brunswick, N.J.) at 150 rpm. Bottles were vented with a sterile needle to release CO₂ prior to weighing for weight loss to monitor ethanol production. Enzymes used were cellulase Spezyme CP (Genencor-Danisco, Beloit, Wis.) at 59 filter paper units (FPU)/mL, and Genencor Accelerase BG β-glucosidase at 25% volume ratio to Spezyme per the manufacturer's recommendations. Enzymes were kindly provided by Genencor International. Experiments were conducted with 30 FPU cellulase per gram rice biomass cellulose. Fermentation performance was determined by HPLC as described (Yang and Wyman, 2004, Biotechnol. Bioeng. 86:88-98) after samples were centrifuged in a Sorvall Biofuge microcentrifuge at 13,000 rpm for 2 min, and the supernatant filtered through a 0.2 um filter to remove solids. Both sugar and ethanol analyses were performed by high performance liquid chromatography (HPLC) using a Biorad Aminex HPX-87H 300_{—}7.8 mm column and a refractive index (RI) detector (Hitachi Model 2490, Pleasanton, Calif.). Ethanol yields were calculated based upon input cellulose and biomass.

### Gene Isolation, Cloning and Transformation of SiPAE2 in Switchgrass.

The foxtail millet (Setaria italica L.) pectin acetylesterase2 (SiPAE2) gene was isolated by screening a foxtail millet fosmid library as described above for rice and the gene was cloned into pANIC 10A vector. Switchgrass line ST1 was transformed with pANIC10A-GA20ox through Agrobacterium-mediated transformation. Embryogenic calli were co-cultivated with Agrobacterium strain EHA105 for three days and then selected on MP medium supplemented with hygromycin 60 mg/L. The resistance calli were regenerated on REG medium and the shoots were rooted on MS medium.

### Genomic PCR of SiPAE2 Over-Expression Switchgrass Lines.

Genomic DNA was isolated according to Lassner et al. (1989, Plant Mol Biol Rep 7:116-128). The transgene was amplified using forward primer 5′ GCGGTGGCCGAACAAGGGAA (SEQ ID NO:23) and reverse primer 5′ CCAGCCGCTCGCATCTTTCCA (SEQ ID NO:24) to give a 576 by product. The reverse primer is located in the AcV5tag of the vector.

### Plant Materials and Growth Conditions of SiPAE2 Over-Expression Switchgrass Lines.

Ten independent switchgrass lines over-expressing the SiPAE2 gene were grown in a growth chamber at 24° C. with 60% constant relative humidity and a 16/8 light/dark cycle photoperiod and fertilized with 14:14:14 NPK once per month.

### Quantitative Real-Time PCR of SiPAE2 Over-Expression Switchgrass Lines.

For transcript analysis, leaves from nine independent transgenic switchgrass line were harvested at E2 stage. Total RNA extraction and cDNA synthesis were carried out as described above for the rice lines. The primers used to amplify the PAE2 transcripts of the above tissue were as follows: PvPAE2 (forward, 5′-ATTTCATGACCTTCTACCACC (SEQ ID NO:25); reverse, 5′-CACTCCAGCAACATCTTTCTC (SEQ ID NO:26)), SiPAE2 (forward, 5′-TCATGCACTTCGATCTTACCC (SEQ ID NO:27); reverse, 5′-TGGCACCAGAATGTTCCTTAC (SEQ ID NO:28)), two primers (forward, 5′-CAAGATTTGGAGATCCCGTG (SEQ ID NO:29); reverse, 5′-AATGCTCCACGGCGAACAG (SEQ ID NO:30)) to amplify the PvActC transcript were also designed as an internal standard for quantification. The subsequent procedural and quantification methods were as described above for SiPAE1 and siPAE2 rice lines.

### Results

SiPAE1 Over-Expression Rice Lines have Increased Growth and Biomass Phenotypes.

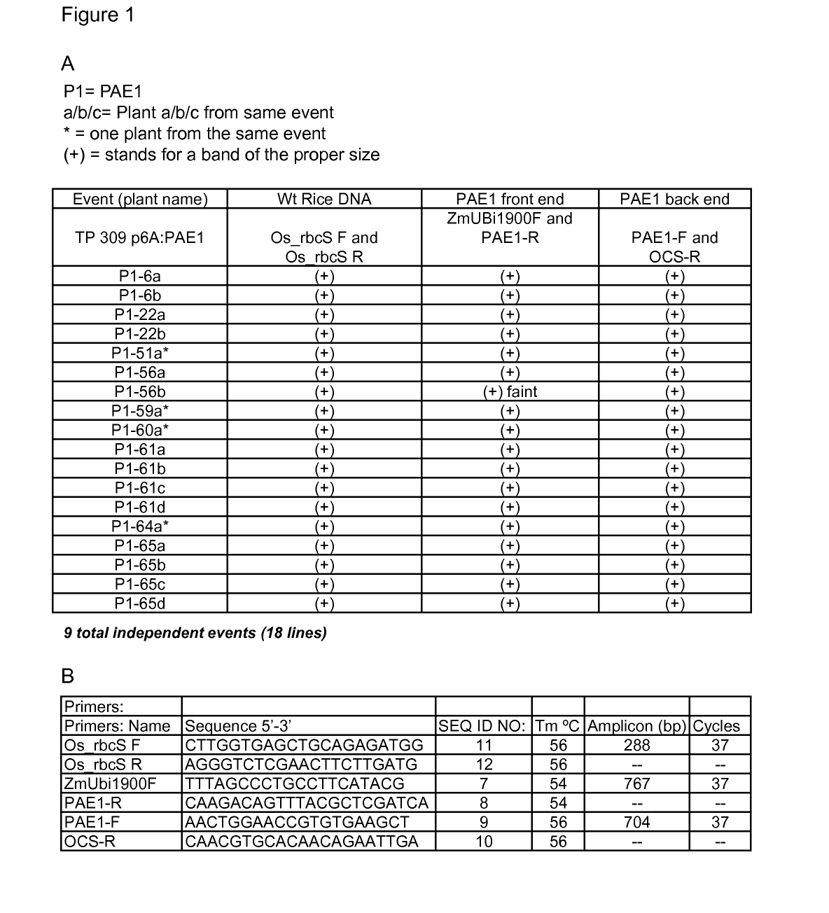

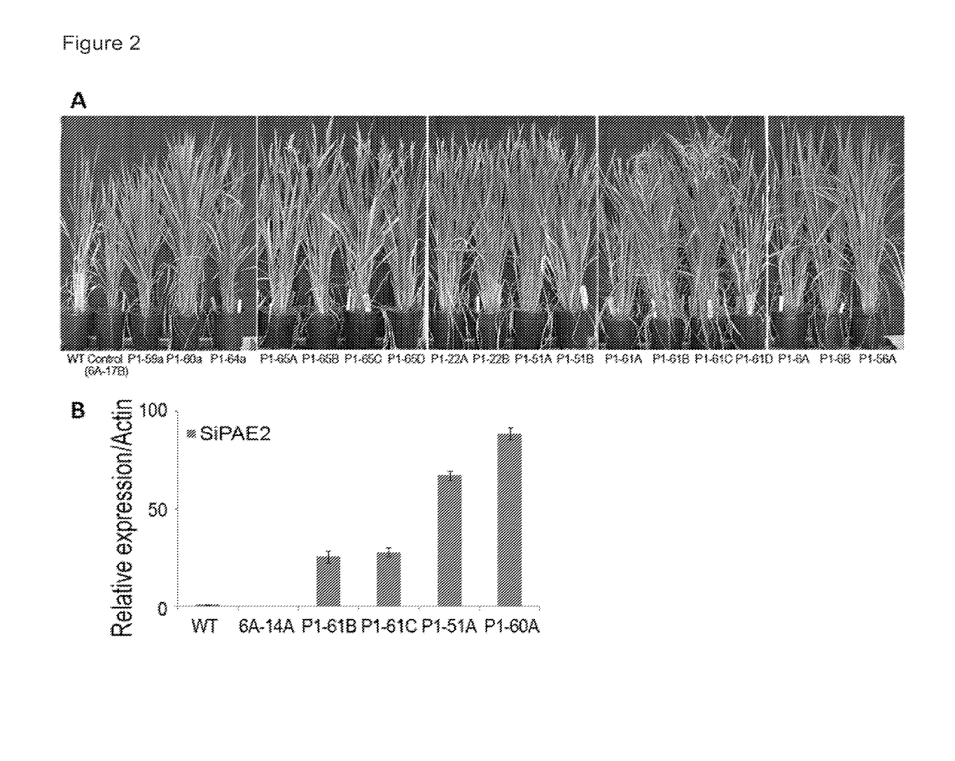

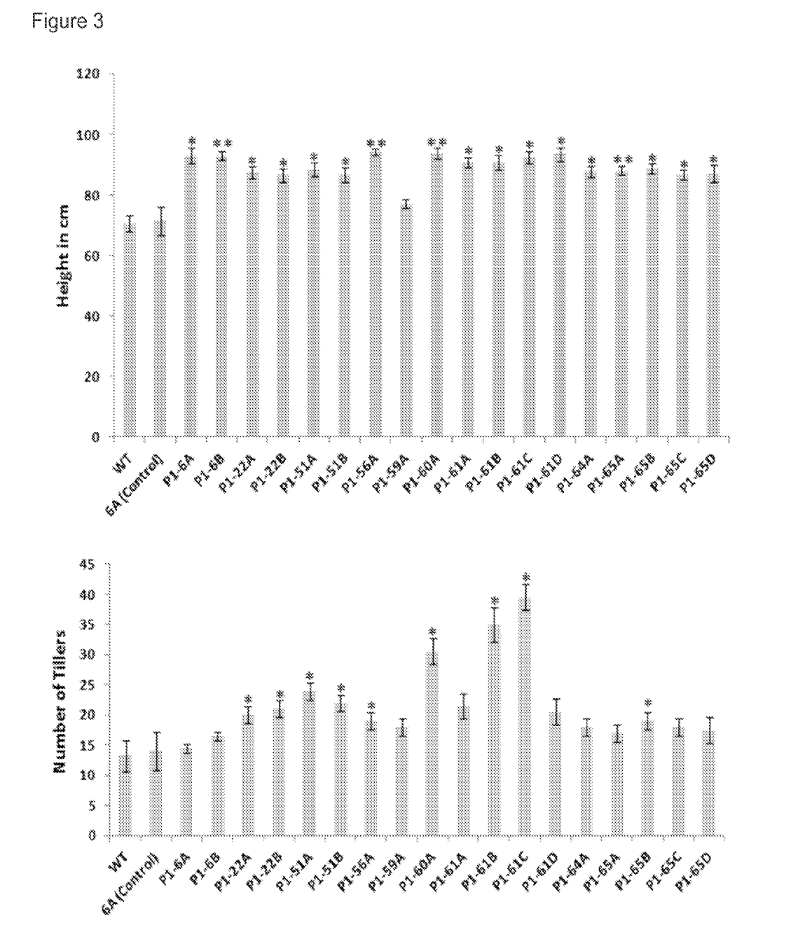

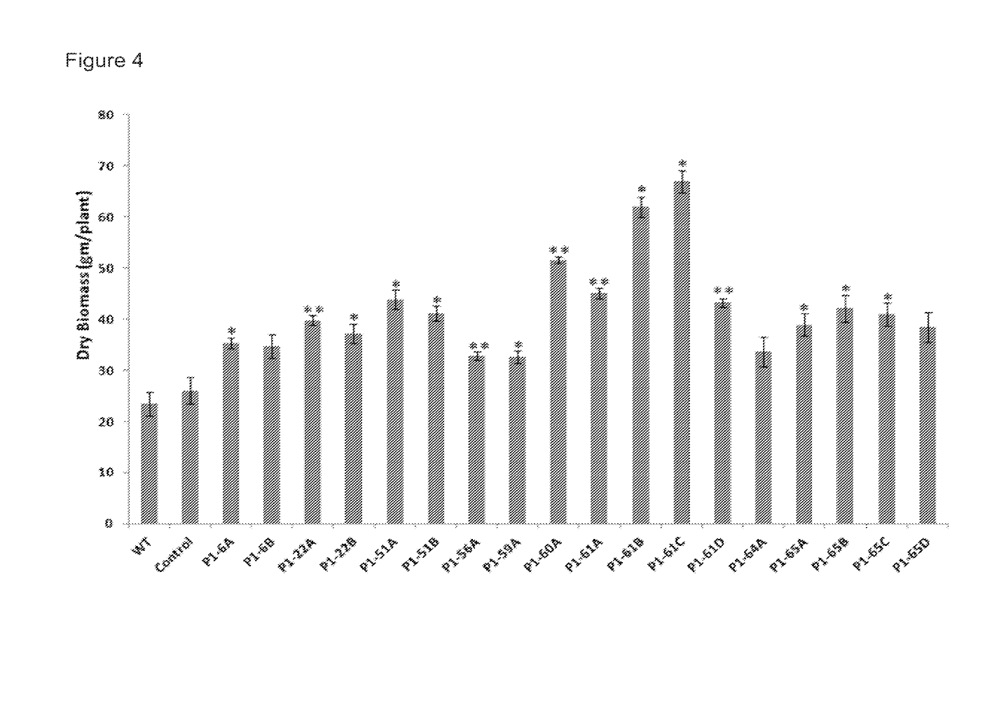

Nine independent events yielding a total of 18 plant lines (P1-6 (a,b); P1-22 (a,b); P1-51a; P1-56 (a,b); P1-59a; P1-60a; P1-61(a-d); P1-64a; P1-65 (a-d)) of SiPAE1over-expression rice lines were confirmed as transgenic by genomic PCR analysis (FIG. 1). After 80 days of growth, seventeen of the eighteen OsPAE1-OE lines had greater plant height and nine lines also had an increased number of tillers in comparison to WT and control (FIGS. 2a and 3). The transcript level of four SiPAE1-OE lines were analyzed and all four lines showed increase transcript levels (FIG. 2b). Similarly, fifteen of the eighteen OsPAE1-OE lines had greater dry weight biomass compared to wild type and control (FIG. 4).

Monossacharide Composition of Cell Walls from SiPAE1 Over-Expression rice Lines.

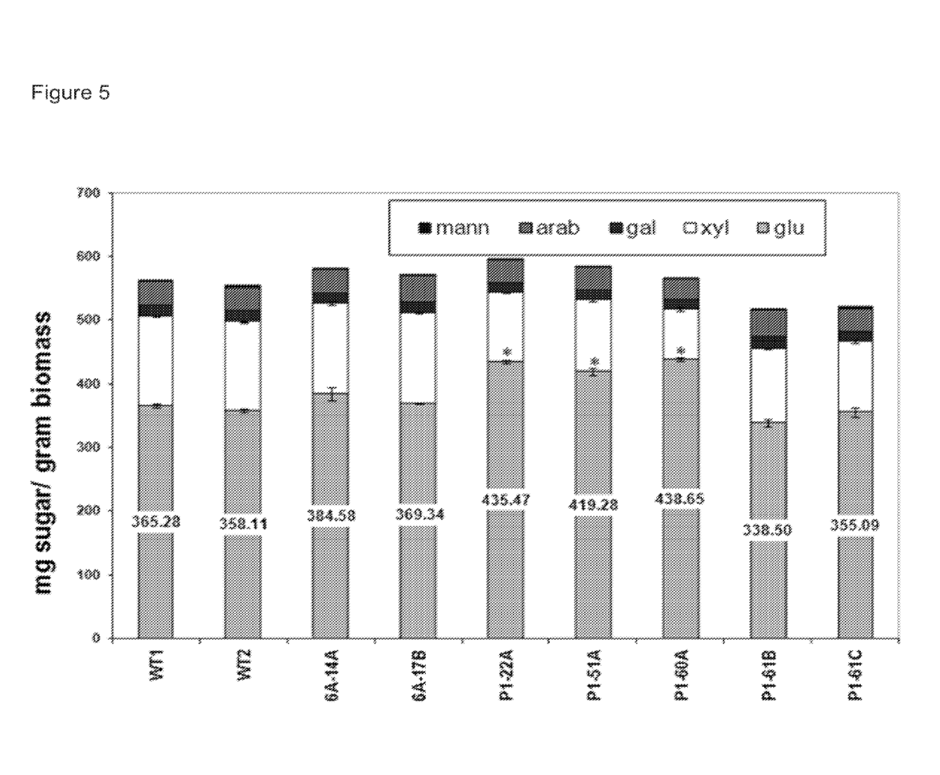

SiPAE1-OE rice transgenic lines P1-22A, P1-51A and P1-60A had a greater glucose and reduced xylose content in comparison to vector control and wild type (FIG. 5).

### Rice SiPAE1 Over-Expression Lines Yield Increased Ethanol Yield Following Fermentation.

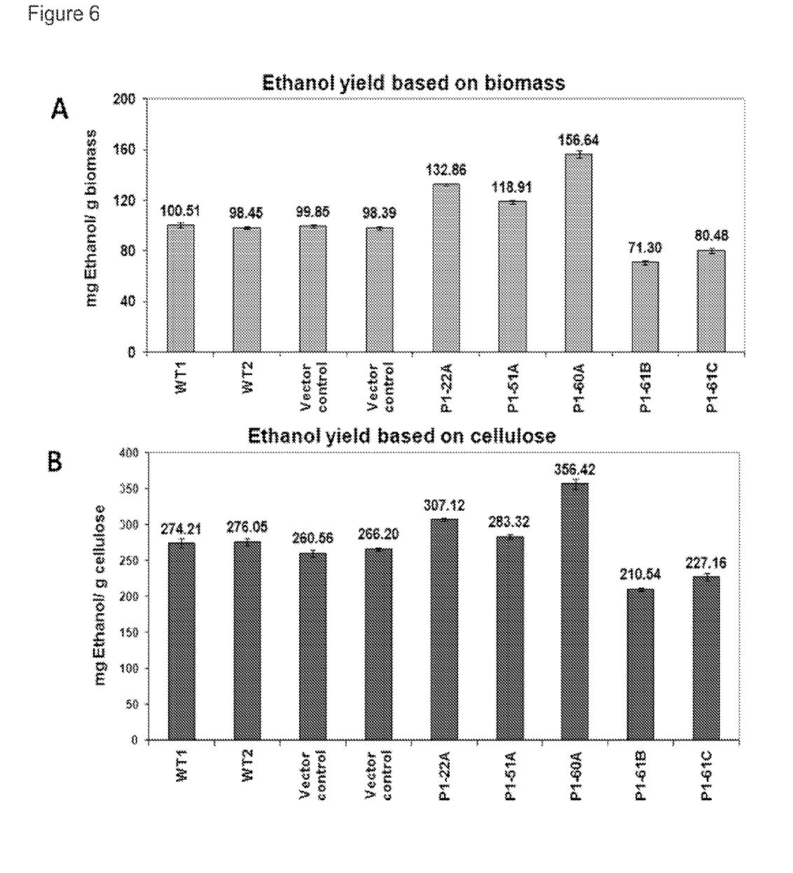

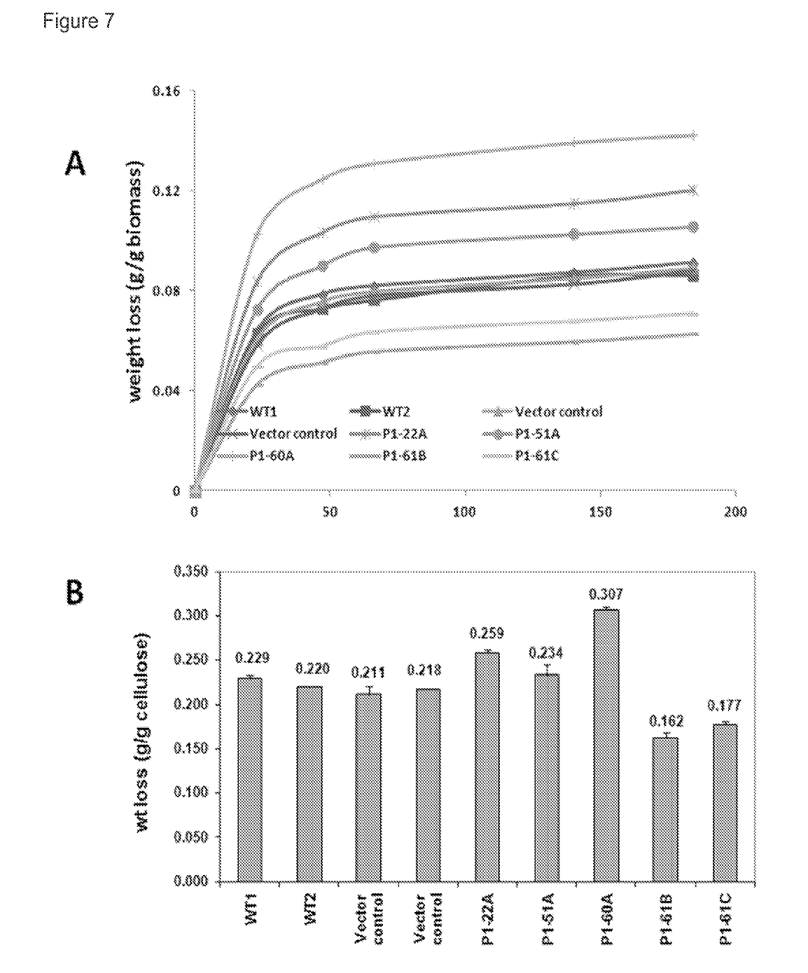

The impact of SiPAE1 over-expression in rice on enzymatic hydrolysis and fermentation of rice biomass was tested. The transgenic rice lines and their corresponding controls were subjected to an initial pretreatment of autoclaving for 30 minutes followed by simultaneous saccharification and fermentation (SSF) to measure the production of ethanol. The transgenic lines had a significant increase in ethanol yield per gram biomass (18%, 32% and 56% for P1-51A, P1-22A and P1-60A lines, respectively) and cellulose content (11% and 29% for P1-22A and P1-60A lines, respectively) (FIG. 6). The time course of fermentation of rice transgenics lines was examined by recording the weight loss caused by CO₂ escape over time (FIG. 7). This plot shows that the rice SiPAE1-OE transgenic lines had a fermentation pattern similar to the control but yielded ethanol more quickly, reaching a higher level by the end of the fermentation.

### Effect of SiPAE2 Over-Expression (OE) in Rice.

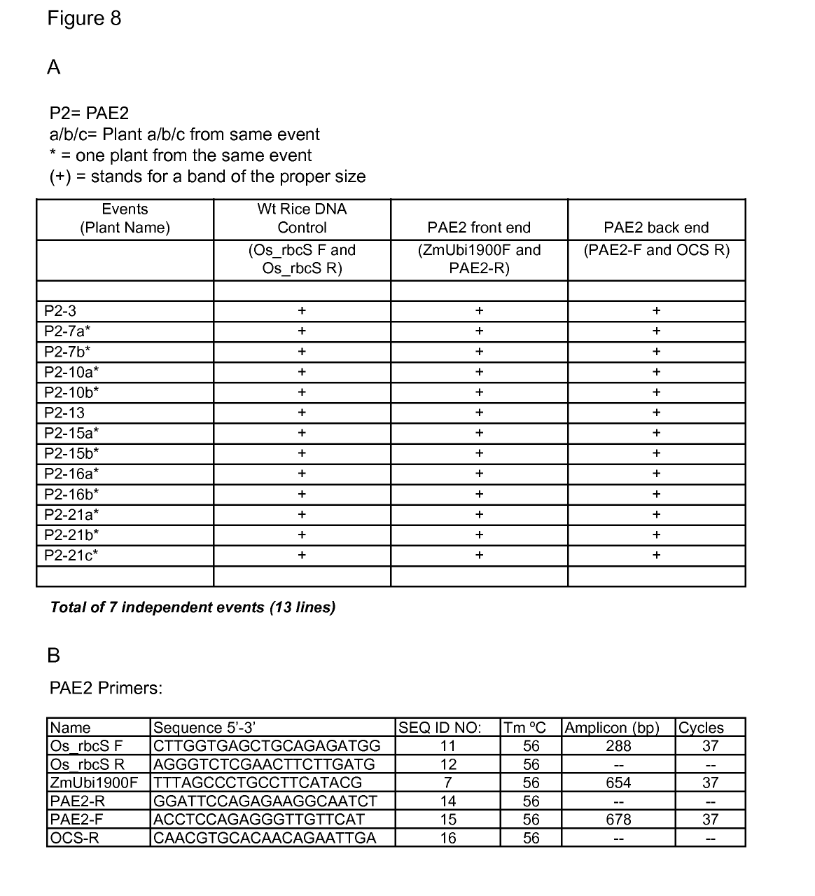

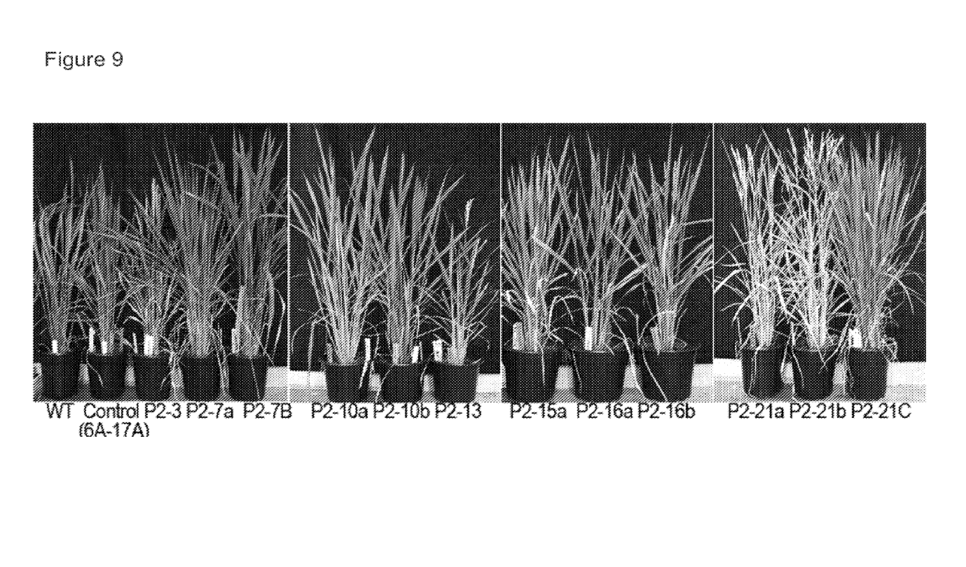

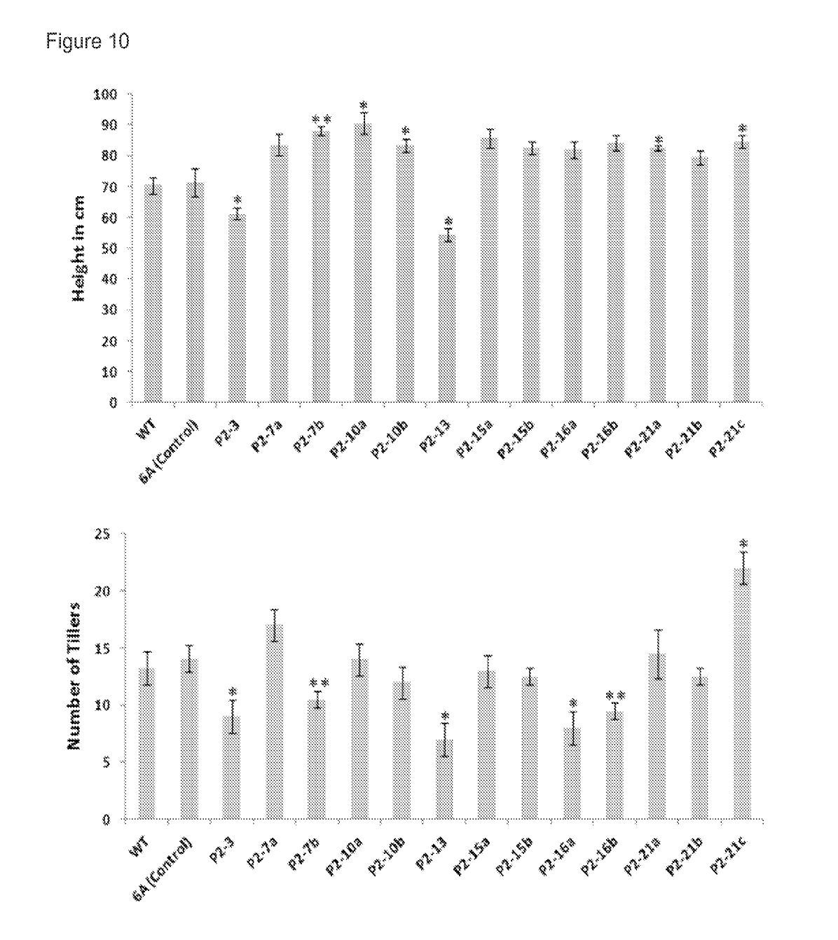

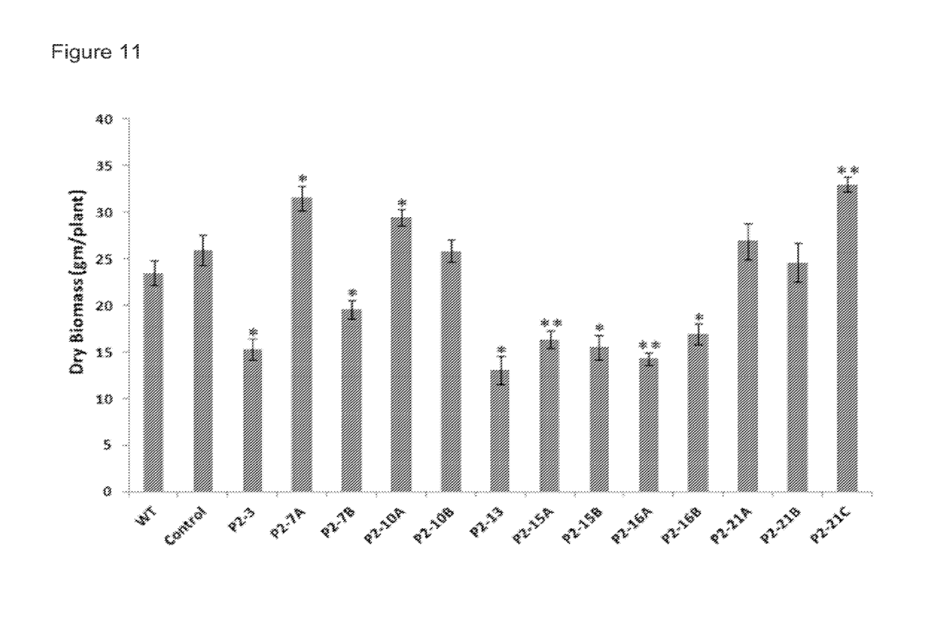

Genomic PCR showed that seven independent events (13 plant lines) of rice SiPAE2 over-expression lines harbored the transgene (FIG. 8). Four of the thirteen rice SiPAE2 over-expression lines had greater plant growth in height and two lines had reduced height in comparison to controls (FIGS. 9 and 10). Similarly, five lines had a decreased number of tillers and one line had an increased number of tiller compared to WT and vector control (FIGS. 9 and 10). Three rice SiPAE2 over-expression lines had greater dry biomass and seven of the thirteen lines had reduced biomass dry weight compared to controls (FIG. 11).

### Effect of SiPAE2 Over-Expression in Switchgrass.

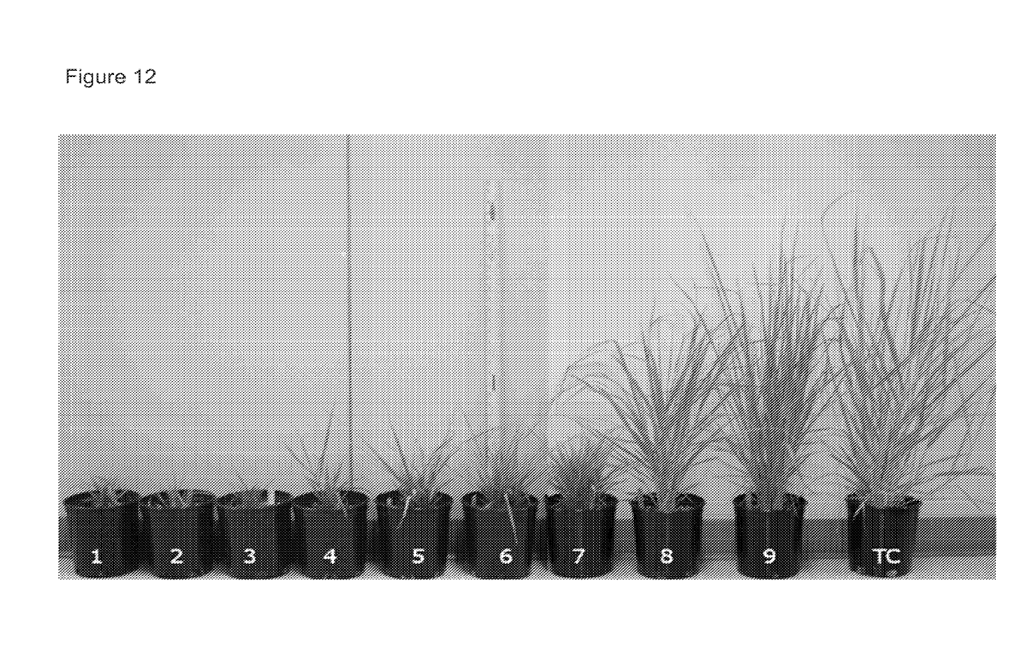

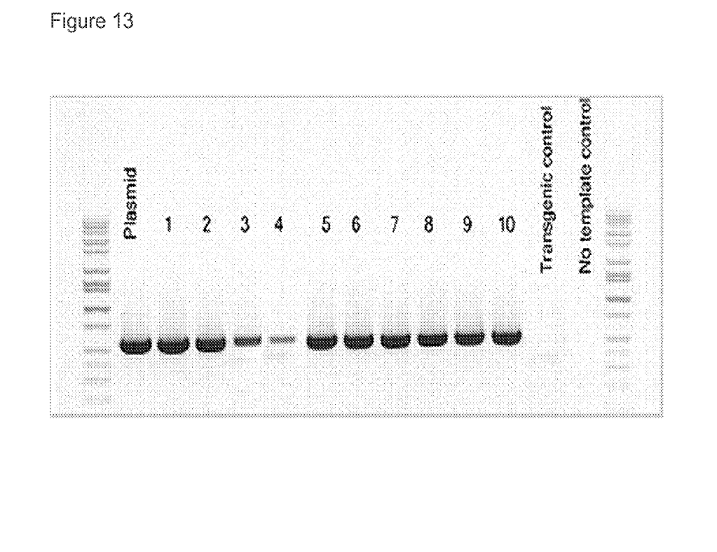

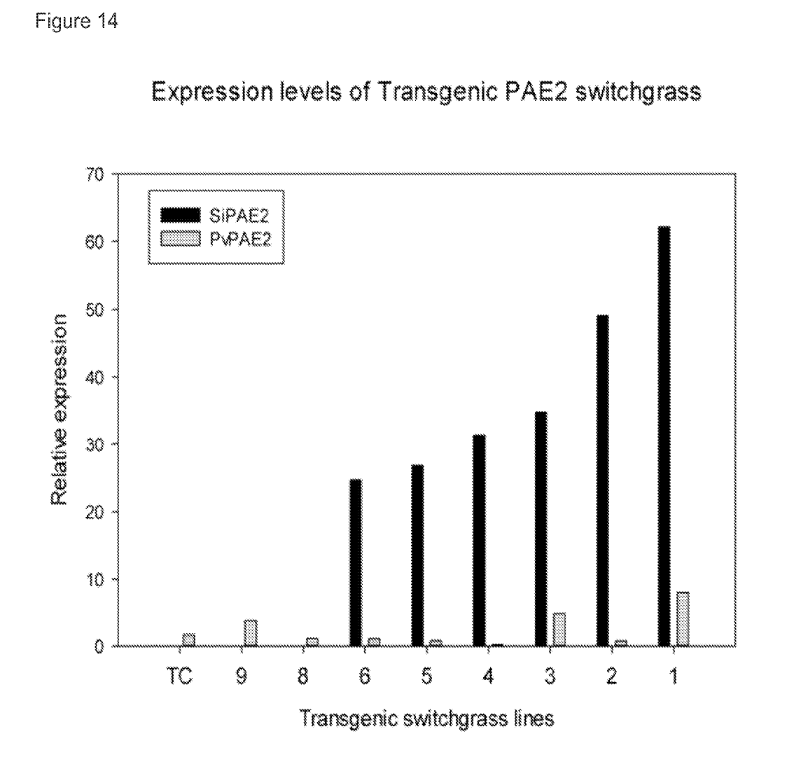

Seven out of ten independent SiPAE2 over-expression transgenic switchgrass events had a severe dwarfing phenotype. The dwarf SiPAE2-OE switchgrass had very short internodes and smaller leaves compared to the taller transgenics and to the control (FIG. 12). The transgenic switchgrass expressing RFP were screened by genomic PCR specific for the transgene. Transgenic switchgrass found to be negative for the PAE2 gene but positive for RFP and hygromycin genes which were used as the control (FIG. 13). All 10 independent transgenic lines contained SiPAE2 as shown in FIG. 14. As expected, the transgenic control was negative for SiPAE2. Six lines showed high transgene expression levels relative to actin gene (FIG. 14). Two lines show lower relative gene expression than the native PAE2. The transgenic control line did not show any amplification for the PAE2 transgene. The levels of native PAE2 varied between the lines.

### Conclusions

Genomic PCR was used to confirm the transgenic rice SiPAE1 over-expression (OE), and switchgrass and rice SiPAE2 over-expression lines. An increase in plant height, number of tillers and dry biomass was found in rice SiPAE1 over-expression lines. The SiPAE1 over-expression in the transgenic rice lines improved ethanol yield by up to 18-56% (depending upon the line) in comparison to the wild type and vector controls. A reduced need for pretreatment severity and reduction in enzymes to obtain high ethanol yields from fermentation can reduce the cost of biomass processing. The 18-56% increase in ethanol yield obtained by over-expression of SiPAE1 in rice could provide a mechanism for improving in ethanol yield in biofeedstocks and reduced energy costs.

An up to 65-fold increase in gene expression was detected in switchgrass transgenic lines overexpressing SiPAE2 with an accompanying severe reduction in plant height. The reduction in plant height corresponding to the increased transcript level in the switchgrass SiPAE2 transgenic plants. Native PAE2 expression varied across the switchgrass transgenic lines. However, the effect on growth of over-expression of SiPAE2 was more pronounced in switchgrass than rice.

The increase in growth and ethanol yield in the rice SiPAE1 over-expression lines and the decreased growth and biomass in switchgrass and rice SiPAE2 over-expression lines indicates that acetylation of pectin significantly affects plant growth and biomass properties in rice and switchgrass, a somewhat unexpected result considering the relatively low amounts of pectin in these grass species. The results also indicate that specific structural modifications of pectin affect grass growth and biomass yield. Finally, the different effects of the over-expression of PAE1 versus PAE2 indicate that these enzymes have unique and functionally distinct roles in grass biomass formation and on biomass properties.

### EXAMPLE 2

### Over-Expression of Foxtail Millet Pectin Acetylesterase 1 (SiPAE1) in Switchgrass

### Materials and Methods

### Gene Isolation, Cloning and Transformation of SiPAE1 in Switchgrass.

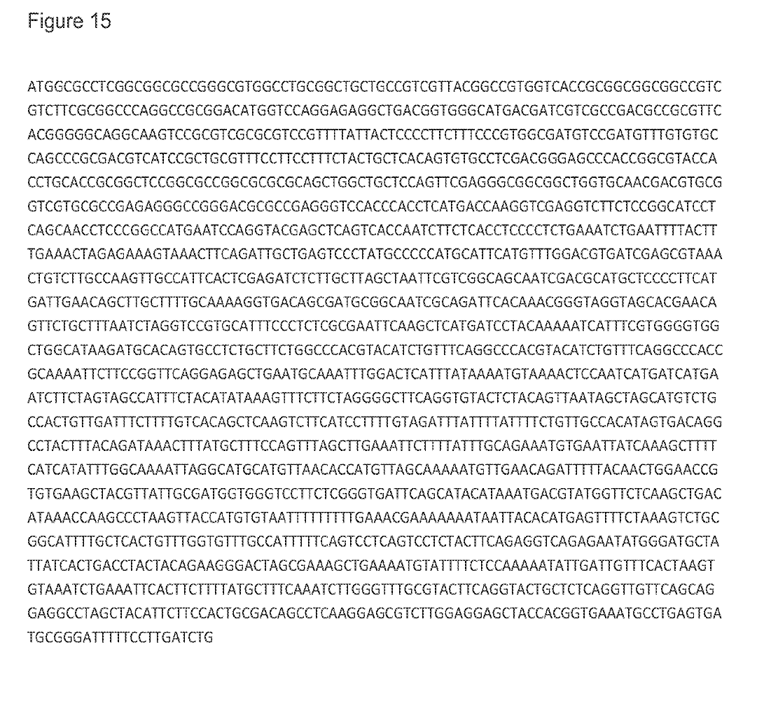

The foxtail millet (Setaria italica L.) pectin acetylesterasel (SiPAE1) gene was isolated by screening a foxtail millet fosmid library as described above, and the gene was cloned into pANIC 10A vector. Switchgrass lines ST1 and ST2 were transformed with pANIC10A-GA20ox, containing the nucleoitde sequence shown in FIG. 15, through Agrobacterium-mediated transformation. Embryogenic calli were co-cultivated with Agrobacterium strain EHA105 for three days and then selected on MP medium supplemented with hygromycin 60 mg/L. The resistance calli were regenerated on REG medium and the shoots were rooted on MS medium.

### Genomic PCR of SiPAE1 Over-Expression Switchgrass Lines.

Genomic DNA was isolated according to Lassner et al (1989, Plant Mol Biol Rep 7:116-128). The transgene was amplified using forward primer 5′TCGATGCTCACCCTGTTGTTTGG (SEQ ID NO:31) and reverse primer 5′CGATCATAGGCGTCTCGCATATCTC (SEQ ID NO:32) to give a 2891 by product. The forward primer is located in the intron of the maize ubiquitin promoter and the reverse primer is located in the nopaline synthase terminator of the vector.

### Plant Materials and Growth Conditions of SiPAE1 Over-Expression Switchgrass Lines.

Five independent switchgrass lines (PAE1-1 and PAE1-2 are from ST2 genotype; PAE1-3, PAE1-4 and PE1-5 are from ST1 genotype) over-expressing the SiPAE1 gene were grown in a growth chamber at 24° C. with 60% constant relative humidity and a 16/8 light/dark cycle photoperiod and fertilized with 14:14:14 NPK once per month.

### Quantitative Real-Time PCR of SiPAE1 Over-Expression Switchgrass Lines.

For transcript analysis, leaves from five independent transgenic switchgrass line were harvested at E2 stage. Total RNA extraction and cDNA synthesis were carried out as described above. The primers used to amplify the PAE1 transcripts of the above tissue were as follows: SiPAE1 (forward, 5′-GATGTCCGATGTTTGTGTGC (SEQ ID NO:19); reverse, 5′-CTCGACCTTGGTCATGAGGT (SEQ ID NO:20)), two primers (forward, 5′-CAAGATTTGGAGATCCCGTG (SEQ ID NO:29); reverse, 5′-AATGCTCCACGGCGAACAG (SEQ ID NO:30)) to amplify the PvActC transcript were also designed as an internal standard for quantification.

### Saccharification.

Biomass was extracted with alpha-amylase (Spirizyme Ultra-0.25%, Novozymes) and beta-glucosidase (Liquozyme SC DS-1.5%, Novozymes) in 0.1 M sodium acetate (24 hours, 55° C., pH 5.0) to remove possible starch content (16 mL enzyme solution per 1 g biomass). This was followed by an ethanol (190 proof) soxhlet extraction for an additional 24 hours to remove extractives. After drying overnight, 5 mg (+/−0.5 mg) was weighed in triplicate into one of 96 wells in a solid hastelloy microtitre plates. Water was added (250 uL), the samples sealed with silicone adhesive, teflon tape, and reacted at 180° C. for 17.5 minutes. Once cooled 40 uL of buffer-enzyme stock was added. The buffer-enzyme stock was 8% CTec2 (Novozymes) in 1 M sodium citrate buffer. The samples were then gently mixed and left to statically incubate at 50° C. for 70 hours. After 70 hours incubation an aliquot of the saccharified hydrolysate was diluted and tested using Megazymes GOPOD (glucose oxidase/peroxidase) and XDH assays (xylose dehydrogenase) (Megazyme International Ireland). Results were calculated using mixed glucose/xylose solutions.

### Results

### Effect of SiPAE1 Over-Expression in Switchgrass.

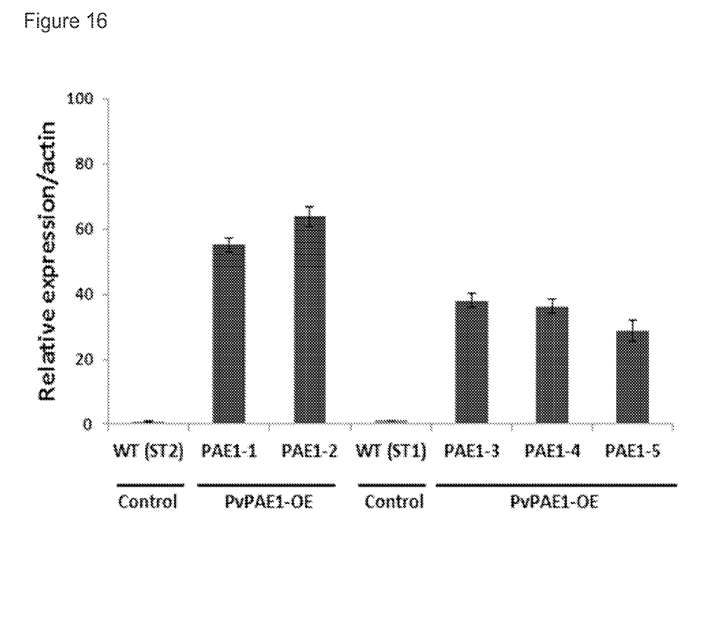

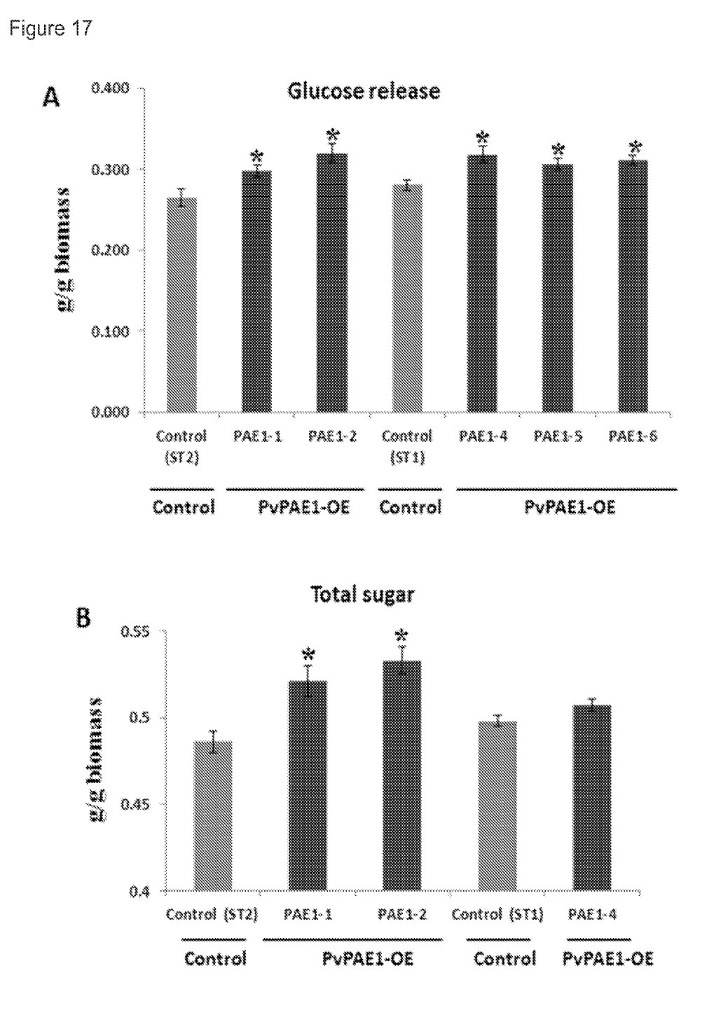

Five independent SiPAE1 over-expression transgenic switchgrass lines (PAE1-1 and PAE1-2 were from ST2 genotype; PAE1-3, PAE1-4 and PE1-5 were from ST1 genotype) were confirmed as transgenic by genomic PCR analysis. After 55 days of growth, transgenic switchgrass found no major change in growth phenotype as compared to their respective wild type control. Five lines showed high transgene expression levels relative to actin gene (FIG. 16).The SiPAE1-OE transgenic switchgrass lines had a significant increase in glucose yield per gram biomass varying from 9 to 21% across the lines (13%, 21%, 13%, 9%, and 11% for PAE1-1, PAE1-2, PAE1-3, PAE1-4 and PAE1-5 lines, respectively) (FIG. 17A). Similarly, total sugar also increased 2 to 10% in three SiPAE1-OE transgenic switchgrass lines in comparison to wild type (7%, 10% and 2% for PAE1-1, PAE1-2 and PAE1-5 lines, respectively) (FIG. 17B).

### Conclusion

Genomic PCR was used to confirm the SiPAE1-OE switchgrass transgenic lines. An increase in gene expression of up to 63-fold was detected in switchgrass transgenic lines overexpressing SiPAE 1. The SiPAE 1 over-expression in the transgenic switchgrass lines improved glucose release yield by up to 9-21% (depending upon the line) in comparison to the wild type. This increase in glucose yield obtained by over-expression of SiPAE1 in swicthgrass could provide a mechanism for improving in ethanol yield in biofeedstocks and reducing energy costs.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1-9. (canceled)

10. A transgenic plant comprising increased expression of a coding region encoding a PAE polypeptide compared to a control plant, wherein the PAE polypeptide is a member of the Carbohydrate-Active Enzymes (CAZy) carbohydrate esterase family 13.

11. The transgenic plant of claim 10 wherein the transgenic plant comprises a phenotype selected from decreased recalcitrance, increased growth, or the combination thereof.

12. The transgenic plant of claim 10 wherein the transgenic plant is a monocot plant.

13. The transgenic plant of claim 12 wherein the transgenic plant is a member of the family Poaceae.

14. (canceled)

15. The transgenic plant of claim 12 wherein the transgenic plant is a member of the subfamily Panicoideae.

16. The transgenic plant of claim 10 wherein the transgenic plant is a woody plant.

17. The transgenic plant of claim 16 wherein the transgenic plant is a member of the genus Populus.

18. A part of the transgenic plant of claim 10 wherein the part is chosen from a leaf, a stem, a flower, an ovary, a fruit, a seed, and a callus.

19. The progeny of the transgenic plant of claim 10.

20. The progeny of claim 19 wherein said progeny is a hybrid plant.

21. Biomass obtained from the transgenic plant of claim 10.

22-24. (canceled)

25. A method for using a transgenic plant, the method comprising processing a transgenic plant to result in a processed pulp, wherein the transgenic plant comprises increased expression of a coding region encoding a PAE polypeptide compared to a control plant, wherein the PAE polypeptide is a member of the Carbohydrate-Active Enzymes (CAZy) carbohydrate esterase family 13.

26. The method of claim 25 wherein the processing comprises a physical pretreatment, a chemical pretreatment, or a combination thereof.

27. The method of claim 25 further comprising hydrolyzing the processed pulp.

28. (canceled)

29. A method for producing a metabolic product comprising:
contacting under conditions suitable for the production of a metabolic product a microbe with a composition comprising a processed pulp obtained from the transgenic plant of claim 10.

30. The method of claim 25 further comprising contacting the processed pulp with an ethanologenic microbe.

31. (canceled)

32. The method of claim 29 further comprising obtaining a metabolic product.

33. (canceled)

34. The method of claim 29 wherein the metabolic product comprises an alcohol selected from ethanol, butanol, a diol, and a combination thereof.

35. The method of claim 32 wherein the metabolic product comprises an organic acid.

36-41. (canceled)

42. The transgenic plant of claim 10 wherein the PAE polypeptide comprises an amino acid sequence having at least 80% identity to amino acids 25-434 of SEQ ID NO:2.
